(19) United States
(12) Patent Application Publication
(10) Pub. No.: US 2026/0182963 A1
(43) Pub. Date: Jul. 2, 2026

(54) SYSTEMS AND METHODS FOR PROVIDING SENSORY OUTPUT INDICATING LUNG THICKENING DUE TO CONGESTION, INFECTION, INFLAMMATION, AND/OR FIBROSIS IN A SUBJECT

(71) Applicant: Bruce KIMURA, Coronado, CA (US)

(72) Inventor: Bruce KIMURA, Coronado, CA (US)

(21) Appl. No.: 19/129,776

(22) PCT Filed: Nov. 7, 2023
(86) PCT No.: PCT/IB2023/061201

§ 371 Date: May 14, 2025

Related U.S. Application Data
(60) Provisional application No. 63/425,029, filed on Nov. 14, 2022.
(60) Provisional application No. 63/463,963, filed on May 4, 2023.

(51) Int. Cl. A61B 8/00; A61B 8/08; G16H 50/20

(52) U.S. Cl. CPC A61B 8/46; A61B 8/08; A61B 8/4227; G16H 50/20

(57) ABSTRACT

Providing sensory output indicating lung thickening due to congestion, infection, inflammation, and/or fibrosis in a subject are described. Sensor output signals are processed to detect areas of lung thickening and a sensory output device is controlled to generate and/or modulate sensory output for spatially localized areas of lung thickening. In some embodiments, the system uses two-dimensional video images from an ultrasound machine and generates an audio signal such as a “click” each time a B-line, the visual representation of lung edema or fibrosis, is detected on the image. During real-time ultrasound imaging of a patient's lung, the patient may hear multiple clicks with varying intensity and frequency depending on proximity to areas of lung thickening. This facilitates examination of the lung using diagnostic ultrasound with feedback that is heard rather than seen, with louder and more frequent clicks representing areas of more diseased lung.

### CROSS-REFERENCE

This application claims the benefit of International Patent Application No. PCT/IB2023/061201, filed Nov. 7, 2023, which claims priority to U.S. Provisional Patent Application No. 63/425,029, filed Nov. 14, 2022, and U.S. Provisional Patent Application No. 63/463,963, filed May 4, 2023, said applications are incorporated herein by reference in their entirety for all purposes.

### BACKGROUND

### 1. Field

The present disclosure relates to the field of diagnostic medical ultrasound.

### 2. Description of the Related Art

Examination of the lung for infection, swelling, or scarring by a physician has traditionally occurred using a stethoscope in which generated sounds, named “rales,” are heard and localized to the affected lobes. Radiographic techniques, including chest X-rays or CT scanning, and/or other techniques such as ultrasound may also be used for this type of examination. However, none of these techniques produce a meaningful audio output during examination that may be used to spatially localize areas of infection, swelling, or scarring.

### SUMMARY

The following is a non-exhaustive listing of some aspects of the present techniques. These and other aspects are described in the following disclosure.

Some aspects include a method for providing sensory output indicating lung thickening in a subject. The method comprises moving a sensor proximate to one or both lungs of the subject. The sensor is configured to generate one or more output signals comprising information indicating areas of lung thickening due to congestion, infection, inflammation, and/or fibrosis. The method comprises generating, with a sensory output device, sensory output indicating the areas of lung thickening as the sensor moves proximate to the lungs. The method comprises executing, with one or more processors configured by machine readable instructions, a trained machine learning model to detect the areas of lung thickening based on the output signals. The method comprises controlling, with the one or more processors, the sensory output device to generate and/or modulate the sensory output at spatially localized areas of lung thickening when the sensor moves proximate to the areas of lung thickening.

In some embodiments, the sensory output is configured to facilitate detection and localization of abnormalities associated with lung thickening in the lungs.

In some embodiments, the sensor comprises an ultrasound apparatus.

In some embodiments, detecting the areas of lung thickening based on the output signals, and controlling the sensory output device to generate and/or modulate the sensory output at the areas of lung thickening, occurs in real time as the sensor moves proximate to the areas of lung thickening in the subject.

In some embodiments, sensor movement proximate to one or both lungs of the subject comprises a back and forth rastering motion across the lungs.

In some embodiments, the sensory output comprises: auditory output including one or more sounds generated by the sensory output device; haptic output including one or more vibrations or patterns of vibrations generated by the sensory output device; and/or visual output including one or more flashes or patterns of flashes of light, and/or one or more colors or patterns of colors of light, generated by the sensory output device. In some embodiments, the sensory output intensifies or otherwise modulates as the sensor moves closer to an area of lung thickening. In some embodiments, the sensory output comprises auditory output, and the auditory output comprises a series of clicks configured to intensify as the sensor moves closer to an area of lung thickening. In some embodiments, intensification comprises an increase in click volume and/or click frequency.

In some embodiments, the one or more output signals comprise, and/or are used to generate, raw radiofrequency data, one or more images of the lungs of the subject, pre and post processed scan lines, and/or a constructed postprocessed 2D image. In some embodiments, the output signal is derived directly from the raw radiofrequency data obtained from the sensor (e.g. an ultrasound transducer) prior to the signal being pre- and post-processed and incorporated into a 2D image.

In some embodiments, the one or more output signals indicate B-lines associated with the lungs of the subject, and the trained machine learning model is trained to detect the areas of lung thickening based on the B-lines, from the characteristics of the radiofrequency scan line data, and/or based on other information.

In some embodiments, the trained machine learning model is trained by: obtaining and providing prior sensor output signals associated with areas of lung thickening to the machine learning model; where portions of the prior sensor output signals associated with the areas of lung thickening are labeled as thickened lung areas. In some embodiments, the trained machine learning model is trained with training data, and the training data comprises input output training pairs comprising a labeled B-line in an output signal and a corresponding indication of spatially localized area of the lungs. In some embodiments, the trained machine learning model is trained based on database ultrasound images that have been ranked according to B-line number and echogenicity, representing diagnostic certainty for an abnormal image such that the more numerous and distinct the B-lines appear on an image the more recognizable the sensory output. In some embodiments, the database ultrasound images comprise extracted individual frames from a video, where an annotation tool has been applied to visualize and/or label relevant properties of a frame, and where, for image classification, relevant properties include an estimated severity score, an annotator's confidence score for the estimated severity score, a number of B-lines, and/or a number of A-lines.

In some embodiments, the trained machine learning model is configured to use temporal characteristics of training data including disappearance of B-lines during respiration and data averaging to assess severity of lung thickening, so as to avoid errors due to evanescence or confluence of B-lines.

In some embodiments, the trained machine learning model is trained on a dataset that is uniquely interpreted cognizant to a finding's relationship to diagnostic certainty, such that a diagnostic accuracy and clinical utility of the system is higher than prior systems.

In some embodiments, the sensor, the sensory output device, and the one or more processors comprise a wearable device, and the wearable device is configured to be worn at or near an area of lung thickening.

In some embodiments, the machine learning model comprises a deep neural network.

In some embodiments, the sensor is configured to be moved by the subject.

Some aspects include a method for proving sensory output indicating an abnormality of interest in a target area in an organ of a subject. The method comprises generating, with a sensor configured to move proximate to the target area in the organ of the subject, one or more output signals comprising information indicating (1) a location of the sensor relative to the target area, and (2) presence of the abnormality of interest at or near the target area. The method comprises generating, with a sensory output device, sensory output indicating (1) the location of the sensor relative to the target area, and (2) the presence of the abnormality of interest at or near the target area as the sensor moves proximate to the organ. The method comprises executing, with one or more processors configured by machine readable instructions, a trained machine learning model to (1) determine the location of the sensor relative to the target area, and (2) detect the presence of the abnormality of interest, based on the output signals. The method comprises controlling, with the one or more processors, the sensory output device to generate and/or modulate the sensory output at or near the organ and/or the target area when the sensor moves proximate to the organ and/or the target area. The generating and/or modulating comprises causing the sensory output to modulate as the sensor is moved nearer to, or further from, the organ and/or the target area, such that the sensory output indicates a location of the target area to the subject. The generating and/or modulating comprises, responsive to the sensor being located proximate to the organ and/or the target area, causing the sensory output to indicate spatially localized areas of the target area with the abnormality of interest when the sensor moves around the target area.

Note that, while a display may be used with one or more of the embodiments described herein, a display is not necessarily required. The sensory output may be an audio, haptic, etc. signal, such that there is no display needed, and the present system locates and/or otherwise recognizes an organ and/or target area by artificial intelligence (AI) deep learning of “landmarks” that define when the sensor is positioned correctly. Having reached the target area, the system is configured to activate a diagnostic sound, haptic, etc. sensory output protocol. In the lung, as one example, the system may evaluate the pleural sliding to perform this functionality. This may be likened to a metal detector type sound used to locate an adequate target area, after which the user (or the system itself) can activate one of many sound, haptic, and/or other feedback protocols to diagnose whatever disease the system is programmed for. In some embodiments, the system may use pre-image signal data and/or other data to perform these operations.

Some aspects include a tangible, non-transitory, machine-readable medium storing instructions that when executed by one or more processors, cause the one or more processors to perform operations including some or all of the operations of the above-mentioned process.

Some aspects include a system, including: a sensor, a sensory output device, one or more processors; and memory storing instructions that when executed by the processors cause the processors to effectuate operations of the above-mentioned process.

### BRIEF DESCRIPTION OF THE DRAWINGS

The above-mentioned aspects and other aspects of the present techniques will be better understood when the present application is read in view of the following figures in which like numbers indicate similar or identical elements:

FIG. 1A provides a schematic illustration of the present system configured to provide sensory output indicating lung thickening in a subject.

FIG. 1B illustrates a subject placing the present system (or a portion thereof) on the subject's chest.

FIG. 2 is a schematic illustration of components of an embodiment of the present system.

FIG. 3 illustrates an ultrasound image that includes a B-line.

FIG. 4 illustrates a subject using the present system to detect areas of lung thickening and show results to a physician using telehealth methods.

FIG. 5 illustrates an example of what the subject may present visually to a physician via the subject's smartphone using the present system.

FIG. 6 is a diagram that illustrates an exemplary computing device in accordance with embodiments of the present system.

FIG. 7 is a flow chart that illustrates a method for providing sensory output indicating lung thickening in a subject.

While the invention is susceptible to various modifications and alternative forms, specific embodiments thereof are shown by way of example in the drawings and will herein be described in detail. The drawings may not be to scale. It should be understood, however, that the drawings and detailed description thereto are not intended to limit the invention to the particular form disclosed, but to the contrary, the intention is to cover all modifications, equivalents, and alternatives falling within the spirit and scope of the present invention as defined by the appended claims. Additional descriptions of one or more embodiments are also included in the Appendix included below.

### DETAILED DESCRIPTION OF CERTAIN EMBODIMENTS

To mitigate the problems described herein, the inventor had to both invent solutions and, in some cases just as importantly, recognize problems overlooked (or not yet foreseen) by others in the field of diagnostic medical ultrasound. The inventor wishes to emphasize the difficulty of recognizing those problems that are nascent and will become much more apparent in the future should trends in industry continue as the inventor expects. Further, because multiple problems are addressed, it should be understood that some embodiments are problem-specific, and not all embodiments address every problem with traditional systems described herein or provide every benefit described herein. That said, improvements that solve various permutations of these problems are described below.

For example, examination of the lung for infection, swelling, or scarring by a physician has traditionally occurred using the stethoscope in which generated sounds (“rales”) are heard and localized manually by the physician to the affected lobes. Physicians are trained from medical school in how to recognize rales during auscultation. Radiographic techniques, including chest X-ray or CT scanning can display specific patterns of lung abnormalities, such as interstitial disease or “ground-glass” infiltrates, which are important to recognize for both initial diagnosis or localization of the cause, and can be further used to follow improvement with treatment. For instance, rales or radiographic findings the lung bases are more likely due to heart failure if bilateral, or pneumonia if unilateral. Findings that extend over the entirety of both lungs likely represent heart failure or diffuse pneumonia or fibrotic process. Findings that are confined to a specific area may represent pneumonia. However, localizing findings using auscultation is time intensive and less sensitive than ultrasound, for example, and radiographic techniques require more resources and expose the patient to radiation. In particular in children, respiratory infections are common and radiation exposure is best minimized, limiting the early outpatient detection of treatable pneumonia.

In addition to minimizing radiation exposure in pediatric evaluations, there are many other adult applications where the present system may be useful. For COVID-19, as one example, the presence of B-lines is prognostic. B-lines typically develop within the first week of infection. Detection of B-lines may be used to risk stratify a patient who needs more attention or treatment to prevent hospitalization. In congestive heart failure, the detection of B-lines is prognostic and would result in more aggressive therapies to prevent hospitalization or death. The presence of B-lines helps to differentiate various causes of shortness of breath from each other, such as COPD/asthma versus heart failure versus pneumonia. Different forms of output of the device may be used during telehealth appointment for direct interpretation by healthcare providers; be subject to simplified recommendations (e.g., “please call your physician”) generated by artificial intelligence methods. In addition, this technology could be employed in community centers, pharmacies, schools or elderly homes to assess for the spread of COVID-19, assess mild shortness of breath, or follow patients after recent hospitalization, for example.

The high content of air within the lung often limits the ultrasound diagnosis to reverberation artifacts generated at the lung surface of the abnormal lung, called B-lines. B-lines are ring-down reverberation artifacts caused by abnormal thickening of the lung surface due to edema or fibrosis, for example, and are present in patients with heart failure, pneumonia, or lung fibrosis. B-lines are distinctive and recognizable typically as 1-10 (vertical or near vertical) streaks on an ultrasound image. B-lines are considered to be one of the simplest of ultrasound findings for a novice user to discover. The presence and number of B-lines have been associated with prognosis in patients, particularly in those with heart failure, one of the most common diagnoses for hospital admission. In heart failure, the reduction of B-lines can occur with proper treatment within minutes. Lung ultrasound has been used in the pediatric population to reduce the use of chest X-rays in the diagnosis of pneumonia. Moreover, B-lines which develop from interstitial edema, may precede findings on auscultation or chest X-ray, techniques that detect a later pathologic stage of alveolar edema. Lungs are more easily imaged with ultrasound for B-lines than auscultated for rales with the stethoscope, particularly in obese individuals. Overall, detection of B-lines is a sensitive technique for detection of edema during the lung examination in patients.

Although a B-line artifact displayed on a two-dimensional ultrasound image is easily seen, the current diagnostic methodology is to count the number of the B-lines and the number of affected lung “zones.” For instance, expert consensus publications have suggested that the presence of two zones in each lung with 3 B-lines within them is considered diagnostic of heart failure. However, this empirically derived threshold is limited for multiple reasons. First, the zones of the lung are simply surface regions on the chest and do not represent specific lung lobes or anatomy. Second, the gradient of findings toward the base of the lungs, characteristic of the effects of gravity on edema, is ignored. Finally, the user has to remember, or record, while imaging which zones have B-lines and how many B-lines are in each of them. Because the mental multi-tasking required is difficult, often during evaluation of an acutely ill patient, newer ultrasound platforms have developed visual maps and recording aides to assist the examiner. These require additional time and training to perform and have not been widely accepted as useful. Furthermore, the clinical utility of localizing the B-line pattern has not been developed for initial diagnosis or in monitoring the disease.

Current use of artificial intelligence methods to detect and count individual B-lines suffers from spatial-temporal resolution issues. At this point in development, the ability for these methods to differentiate transient or fused B-lines (such as “white lung”) is limited as compared to an expert reader's interpretation and the database used for training the algorithm.

Advantageously, the present systems and methods provide sensory output indicating lung thickening due to congestion, infection, inflammation, and/or fibrosis in a patient and/or other subjects. Sensor output signals are processed to detect areas of lung thickening and a sensory output device is controlled to generate and/or modulate sensory output for spatially localized areas of lung thickening. In some embodiments, the system uses two-dimensional video images from an ultrasound machine and generates an audio signal such as a “click” each time a B-line, the visual representation of lung edema or fibrosis, is detected on the image. During real-time ultrasound imaging of a patient's lung, the patient may hear multiple clicks with varying intensity and frequency depending on proximity to areas of lung thickening. This facilitates examination of the lung using diagnostic ultrasound with feedback that is heard rather than seen, with louder and more frequent clicks representing areas of more diseased lung.

The present systems and methods include and/or utilize a trained machine learning model configured to use temporal characteristics of training data including, for example, disappearance of B-lines during respiration, and data averaging, to assess severity of lung thickening, so as to avoid errors due to evanescence or confluence of B-lines. The trained machine learning model is trained on a dataset that is uniquely interpreted cognizant to a finding's relationship to diagnostic certainty, such that a diagnostic accuracy and clinical utility of the system is higher than prior systems and/or manual methods.

The present systems and methods utilize a user's (e.g., a physician's, a patient's, and/or other subject's) natural ability to localize and recognize severity through perception (e.g., sight, sound, vibration) akin to a physician's use of a stethoscope, for example. Similar to metal detectors on the beach or a Geiger counter, the human brain's ability to recognize sights (e.g., colored and/or flashing lights), sounds (e.g., clicks of varying frequency and/or intensity), vibrations, and/or other perceptible stimulants for localization is advantageous, specifically when multi-tasking in stressful situations. Additionally, the human brain quickly recognizes, remembers and classifies patterns of visual, auditory, tactile, etc., data in different regions of the cerebrum, as evidenced by language or music memory, for example. Therefore, light, sound, vibration, and/or other stimulant production during examination can be used in diagnosis. No process or software exists that produces a meaningful visual, audio, tactile, etc., output during or for lung ultrasound, despite that fact that ultrasound platforms can produce an audio and/or other signals. A potential exists to create a new field of medical diagnostics related to sensory (e.g., sight, sound, tactile, etc.) representation of ultrasound data with the present systems and methods.

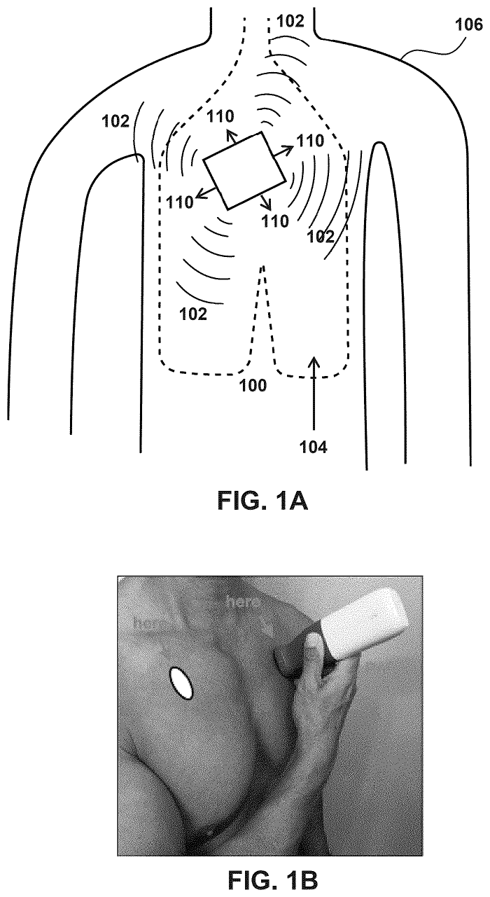

FIG. 1A provides a schematic illustration of a system 100 configured to provide sensory output 102 indicating lung 104 thickening in a subject 106. FIG. 1B illustrates a subject placing system 100 (or a portion thereof) on the subject's chest. Lung thickening may be due to congestion, infection, inflammation, fibrosis, and/or may have other causes. System 100 is configured to move 110 (or be moved) proximate to one or both lungs 104 of subject 106. Movement 110 proximate to one or both lungs 104 of subject 106 comprises a back and forth rastering motion across the lungs, for example, and/or any other movement of system 100 in proximity to one or both lungs 104. Sensory output 102 may include sounds, lights, vibrations, and/or other sensory output. Subject 106 may be a patient being treated by a medical services provider, for example, and/or any other person. System 100 may be moved by a physician, by the subject themself, by a machine, and/or by other methods. System 100 and/or one or more individual components of system 100 (as described below) may have a size and/or a shape that allows system 100 to be held and/or moved by a user (such as subject 106, a physician, and/or other users). Sensory output 102 from system 100 is configured to allow a user to spatially localize thickened areas of lungs 104.

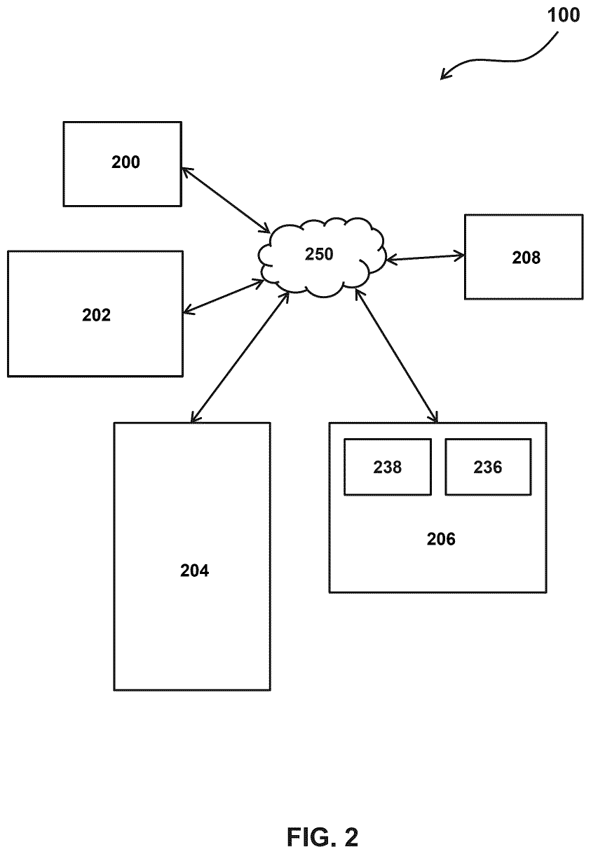

FIG. 2 illustrates an embodiment of system 100 comprising a sensor 200, a sensory output device 202, one or more processors 204, one or more computing devices 206, external resources 208, a network 250, and/or other components. Each of these components is described in turn below.

Sensor 200 is configured to move (e.g., movement 110 shown in FIG. 1A) proximate to one or both lungs (e.g., lungs 104 shown in FIG. 1A) of a subject (e.g., subject 106 shown in FIG. 1A). Sensor 200 is configured to generate one or more output signals comprising information indicating areas of lung thickening due to congestion, infection, inflammation, fibrosis, and/or other causes. In some embodiments, sensor 200 comprises an ultrasound apparatus and/or other sensors. The ultrasound apparatus may include one or more ultrasound transducers configured to obtain ultrasound images of a subject's lungs, for example. An output signal from an ultrasound apparatus may comprise an electronic signal comprising information indicative of the features of a subjects lungs. In some embodiments, the one or more output signals comprise, and/or are used to generate, raw ultrasound/radiofrequency data, one or more images of the lungs of the subject, pre and post processed scan lines, a constructed postprocessed 2D image, and/or other information. In some embodiments, the ultrasound apparatus is configured to obtain an ultrasound image set, a video, and/or other information for the lungs of the subject. In some embodiments, an ultrasound image set or video includes multiple ultrasound images and/or video captured from different angles (e.g., a top view, side view, bottom view, etc.). In some embodiments, for example, the one or more output signals may be used to generate the ultrasound images, which may indicate B-lines associated with the lungs of the subject.

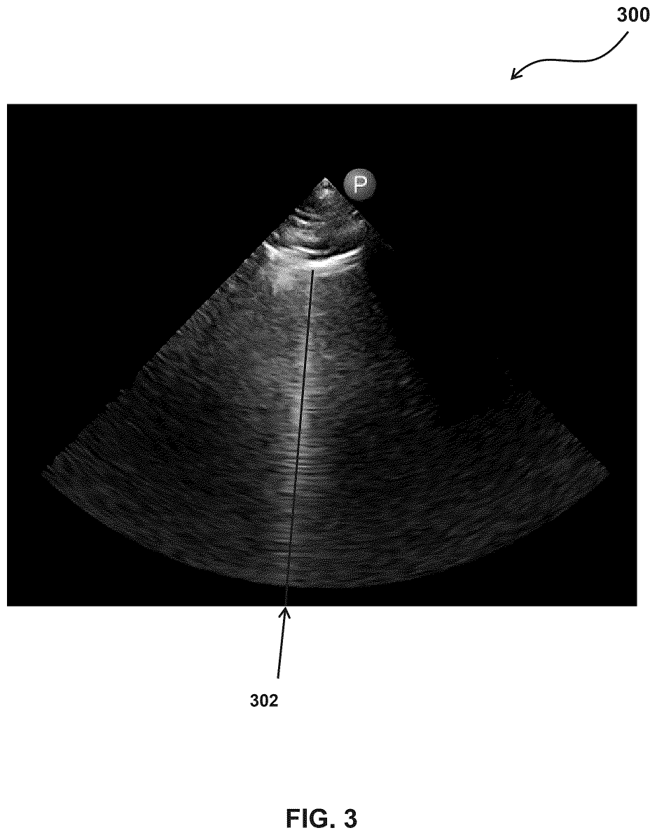

For example, FIG. 3 illustrates an ultrasound image 300 that includes a B-line 302. One or more B-lines may be present in a given ultrasound image 300. The quantity, position, intensity, and/or other properties of a B-line may change over time as an ultrasound is performed, for example. Unlike prior systems that require>=3 B-lines in an image to be “abnormal”, system 100 is configured to display a sound, light, haptic output, etc., representative of the amount of B-lines, either as exceeding a user-specific threshold (e.g., n>=3 or some other threshold) and/or as proportionate to the number of B-lines present (e.g., three clicks for three B-lines). As B-lines move with lung motion, the B-line number may best be obtained at the end of expiration when the lung is still. In order to detect early stages of swelling, the output of system 100 can be compared to the lesser output present during inspiration.

Returning to FIG. 2, in some embodiments, sensor 200 is configured to generate one or more output signals conveying information related to an orientation, movement, position, and/or other characteristics of sensor 200. The information may include and/or be related to an angular position (e.g., a tilt), a spatial position (e.g., in three-dimensional space), a rotation, an acceleration, a velocity, and/or other parameters. Sensor 200 may be configured to operate continuously, at predetermined intervals, and/or at other times before, during, and/or after movement proximate to the lungs of a subject. Sensor 200 may include a chip based sensor included in a surface of sensor 200. Sensor 200 may include accelerometers, gyroscopes, GPS and/or other position sensors, force gages, and/or other sensors. This information may be used by processor(s) 204 (described below) to determine a location (e.g., relative to a subject's chest), movement, and/or other information about sensor 200 and/or system 100. This information may be used to control sensory output device 202 (as described below), and/or for other purposes.

Sensory output device 202 is configured to generate sensory output (e.g., sensory output 102 shown in FIG. 1A) indicating the areas of lung thickening as sensor 200 moves (e.g., movement 110 shown in FIG. 1A) proximate to the lungs (e.g., lungs 104 of subject 106 shown in FIG. 1A). Sensory output device 202 is configured to provide sensory output to a subject, a physician, and/or other users. Sensory output device 202 is configured to provide auditory, visual, somatosensory, electric, magnetic, and/or other sensory output. The auditory, electric, magnetic, visual, somatosensory, and/or other sensory output may include auditory output, visual output, somatosensory output, electrical output, magnetic output, tactile output, a combination of different types of output, and/or other output. The auditory, electric, magnetic, visual, tactile, somatosensory, and/or other sensory stimuli include odors, sounds, visual stimulation, vibrations, somatosensory stimulation, electrical, magnetic, and/or other stimuli. Examples of sensory output device 202 may include one or more of a sound generator, a speaker, a music player, a tone generator, a vibrator (such as a piezoelectric member, for example) to deliver vibratory output, a coil generating a magnetic field, one or more light generators or lamps, one or more light emitting diodes, a fragrance dispenser, an actuator, and/or other devices. The sensory output may have an intensity, a timing, and/or other characteristics that vary as sensor 200 and/or system 100 move toward and/or away areas of lung thickening. In some embodiments, sensory output device 202 is configured to adjust the intensity, timing, and/or other parameters of the stimulation provided to a subject (e.g., as described below) based on the proximity of sensor 200 and/or system 100 to an area of lung thickening.

For example, the sensory output is configured to facilitate detection and localization of abnormalities associated with lung thickening in the lungs. In some embodiments, the sensory output comprises auditory output including one or more sounds generated by sensory output device 202, haptic output including one or more vibrations or patterns of vibrations generated by sensory output device 202, visual output including one or more flashes or patterns of flashes of light, and/or one or more colors or patterns of colors of light, generated by sensory output device 202, and/or other sensory output. In some embodiments, the sensory output intensifies or otherwise modulates as sensor 200 moves closer to an area of lung thickening. As a more specific example, the sensory output may comprise auditory output. The auditory output may comprise a series of clicks configured to intensify as sensor 200 moves closer to an area of lung thickening. In this example, intensification may include an increase in click volume and/or click frequency, and/or other increases in intensity.

One or more processors 204 are configured to provide information processing capabilities in system 100. As such, processor(s) 204 may include one or more of a digital processor, an analog processor, a digital circuit designed to process information, an analog circuit designed to process information, a state machine, and/or other mechanisms for electronically processing information. In some embodiments, a processor 204 may be included in and/or otherwise operatively coupled with sensor 200, sensory output device 202, computing device 206, and/or other components of system 100. Although one or more processors 204 are shown in FIG. 2 as a single entity, this is for illustrative purposes only. In some implementations, processor(s) 204 may include a plurality of processing units. These processing units may be physically located within the same device (e.g., sensor 200, sensory output device 202, computing device 206, etc.), or processor(s) 204 may represent processing functionality of a plurality of devices operating in coordination (e.g., a processor located within sensor 200 and a second processor located within computing device 206). Processor(s) 204 may be configured to execute one or more computer program components. Processor(s) 204 may be configured to execute the computer program component by software; hardware; firmware; some combination of software, hardware, and/or firmware; and/or other mechanisms for configuring processing capabilities on processor(s) 204.

Processor(s) 204 are configured to execute a trained machine learning model to detect the areas of lung thickening based on the output signals. In some embodiments, processor(s) 204 are configured to cause a machine learning model to be trained using training information. In some embodiments, the machine learning model is trained by providing the training information as input to the machine learning model. In some embodiments, the machine learning model may be and/or include mathematical equations, algorithms, plots, charts, networks (e.g., neural networks), and/or other tools and machine learning model components. For example, the machine learning model may be and/or include one or more neural networks having an input layer, an output layer, and one or more intermediate or hidden layers. In some embodiments, the one or more neural networks may be and/or include deep neural networks (e.g., neural networks that have one or more intermediate or hidden layers between the input and output layers).

As an example, neural networks may be based on a large collection of neural units (or artificial neurons). Neural networks may loosely mimic the manner in which a biological brain works (e.g., via large clusters of biological neurons connected by axons). Each neural unit of a neural network may be connected with many other neural units of the neural network. Such connections can be enforcing or inhibitory in their effect on the activation state of connected neural units. In some embodiments, each individual neural unit may have a summation function that combines the values of all its inputs together. In some embodiments, each connection (or the neural unit itself) may have a threshold function such that a signal must surpass the threshold before it is allowed to propagate to other neural units. These neural network systems may be self-learning and trained, rather than explicitly programmed, and can perform significantly better in certain areas of problem solving, as compared to traditional computer programs. In some embodiments, neural networks may include multiple layers (e.g., where a signal path traverses from front layers to back layers). In some embodiments, back propagation techniques may be utilized by the neural networks, where forward stimulation is used to reset weights on the “front” neural units. In some embodiments, stimulation and inhibition for neural networks may be more free flowing, with connections interacting in a more chaotic and complex fashion.

As described above, the trained neural network may comprise one or more intermediate or hidden layers. The intermediate layers of the trained neural network include one or more convolutional layers, one or more recurrent layers, and/or other layers of the trained neural network. Individual intermediate layers receive information from another layer as input and generate corresponding outputs. In some embodiments, the trained neural network may comprise a deep neural network comprising a stack of convolution neural networks, followed by a stack of long short term memory (LSTM) elements, for example. The convolutional neural network layers may be thought of as filters, and the LSTM layers may be thought of as memory elements that keep track of data history, for example. The deep neural network may be configured such that there are max pooling layers which reduce dimensionality between the convolutional neural network layers. In some embodiments, the deep neural network comprises optional scalar parameters before the LSTM layers. In some embodiments, the deep neural network comprises dense layers, on top of the convolutional layers and recurrent layers. In some embodiments, the deep neural network may comprise additional hyper-parameters, such as dropouts or weight-regularization parameters, for example.

In some embodiments, the trained machine learning model is trained by obtaining and providing prior sensor output signals associated with areas of lung thickening to the machine learning model. Portions of the prior sensor output signals associated with the areas of lung thickening are labeled as thickened lung areas. For example, the trained machine learning model may be trained with training data comprising input output training pairs comprising a labeled B-line in an output signal and/or a corresponding ultrasound image generated based on the output signal and a corresponding indication of a spatially localized area of the lungs. In some embodiments, the trained machine learning model is trained to detect the areas of lung thickening based on the B-lines.

For example, the trained machine learning model may be trained based on database ultrasound images that have been ranked according to B-line number and echogenicity, representing diagnostic certainty for an abnormal image such that the more numerous and distinct the B-lines appear on an image the more recognizable the sensory output. The database ultrasound images may comprise extracted individual frames from a video, for example, where an annotation tool has been applied to visualize and/or label relevant properties of a frame. For image classification, relevant properties may include an estimated severity score, an annotator's confidence score for the estimated severity score, a number of B-lines, the presence of A-lines and/or other information. A-lines are evenly spaced, horizontal ultrasound artifacts seen in the normal state as the ultrasound interacts with the pleural surface. As they are horizontal, they are clearly different than the (generally) vertical B-line. The number of A-lines is not important. In severe cases of numerous B-lines, the A-line artifact can disappear completely. In lesser cases, both A and B-lines can be present. The normal has A-lines present.

The trained machine learning model is configured to use temporal characteristics of training data including disappearance of B-lines during respiration and data averaging to assess severity of lung thickening, so as to avoid errors due to evanescence or confluence of B-lines. The trained machine learning model is trained on a dataset that is uniquely interpreted cognizant to a finding's relationship to diagnostic certainty, such that a diagnostic accuracy and clinical utility of system 100 is higher than prior systems.

In some embodiments, a machine learning framework utilized by system 100 and/or processor(s) 204 comprises data labeling, model training, model usage, output post processing, and/or other operations. Data labeling may include providing an input video and/or image set, extracting individual frames (images) from the video and/or images from the image set, and using an annotation tool to visualize/label relevant properties to train on for each frame. For image classification, relevant properties can include an estimated severity score, the annotator's confidence score for this estimated severity score, the number of B-lines, and the number of A-lines, etc., (as described above). Model training may include selecting a model architecture to train on for image classification, such as EfficientNetV2; providing images and corresponding labels for training; and training the model. Model usage may include providing a (new) input video, image set, and/or other stream of data (e.g., an output signal directly from sensor 200); extracting individual frames (images) from the video, set of images, and/or data stream; and running the trained model on each frame to capture predictions. Output post processing may include providing severity and confidence scores; and generating audio (e.g., tone sequences) for these severity and confidence scores. This is configured to provide an audio representation (in this example) for the model's predictions to be heard as an alternative/complementary signal to ultrasound imaging for diagnosing severity. As a proof of concept, a 1:1 association between severity scores and tone sequences was created as a pre-requisite for this step. The tone sequences were generated programmatically, but they could be replaced by audio samples as well.

In some embodiments, this framework may be applied for object detection and semantic/instance segmentation. Instead of labeling estimated severity/confidence scores, bounding boxes and/or polygons can be drawn around B-lines and/or A-lines. A model could then be trained to localize and classify B-lines and/or A-lines. The model could then be used to capture the total number of B-lines and/or A-lines from the detected instances which could be used as an alternative signal for the severity score provided by the image classification model. In some embodiments, a regression model could also be trained instead of a classification model for predicting estimated severity scores and the annotator's confidence score for these estimated severity scores. In some embodiments, input (training) data for the model is not limited to images. The model may also be trained on the raw sensor data used to generate the ultrasound images for optimization purposes (faster inference times, less intermediate processing necessary, more real time usage). Data may still be labeled using ultrasound images, but the raw data associated with these ultrasound images may be used instead of the ultrasound images themselves during training/inference.

One or more processors 204 are configured to control sensory output device 202 to generate and/or modulate the sensory output at spatially localized areas of lung thickening when sensor 200 moves proximate to the areas of lung thickening. Control may comprise electronic communication of one or more commands to sensory output device 202, and/or other control operations. Processor(s) 204 are configured to cause sensory output device 202 to provide sensory output to the subject, a physician, and/or other users, based on a detected area of lung thickening and/or other information. Processor(s) 204 are configured such that controlling sensory output device 202 to provide the sensory the sensory output comprises causing sensory output device 202 to generate and/or modulate (e.g., as described herein), an amount, a timing, and/or intensity of the sensory output at spatially localized areas of lung thickening when sensor 200 moves proximate to the areas of lung thickening. Modulation may comprise changing a timing and/or intensity of the sensory output. In some embodiments, one or more processors 204 are configured such that detecting the areas of lung thickening based on the output signals, and controlling sensory output device 202 to generate and/or modulate the sensory output at the areas of lung thickening, occurs in real time as sensor 200 moves proximate to the areas of lung thickening in the subject.

In some embodiments, one or more processors 204 are configured to cause sensory output device 202 to adjust a volume of sensory output, assign thresholds and/or distinctive sounds for sensory output, record the sensory output, and/or facilitate other functionality. For example, instead of an audio output, during clinical moments in which no sound is desired, such as during patient's sleep, or in loud environments, sensory output device 202 may be controlled such that a color can be displayed on a display screen (e.g., of sensory output device 202, of a computing device 206, etc.), assigned to a threshold finding (e.g., red for abnormal, green for normal; or go from green to yellow to red; etc.).

As a non-limiting example of many of the operations described above, in some embodiments, processor(s) 204 are configured to recognize video output associated with a specific finding, a B-line, on an ultrasound image, and generate an acoustic signal, which has variable tone or frequency related to the severity and/or proximity of findings. System 100 (including an ultrasound sensor for example) allows a user to hear a click each time a B-line is detected. The use of an audio representation facilitates detection and localization of abnormalities in the lung. In some embodiments, two-dimensional video images from an ultrasound sensor are used to generate an audio signal such as a click each time a B-line, the visual representation of lung edema or fibrosis, is detected on the image. As described above, system 100 (processor(s) 204) recognize and assigns weights to a severity of B-line findings using aspects of the acoustic signal.

In some embodiments, system 100 is configured to produce a clinically meaningful acoustic and/or other sensory output signal through deep learning algorithms using neural networks that assign patterns of B-lines displayed on an image an acoustic and/or other sensory output signal. A neural network may be trained using database images that have been ranked according to B-line number and echogenicity, representing diagnostic certainty for an abnormal image. Therefore, the more numerous and distinct the B-lines appear on an image the more recognizable (e.g., more numerous or louder) will be the acoustic signal (and/or other sensory output).

The trained neural network uses temporal characteristics of ultrasound training data such as disappearance of B-lines during respiration, and data averaging, to help assess severity, so as to avoid errors due to evanescence or confluence of B-lines. By training the neural network on a dataset that is uniquely interpreted cognizant to the finding's relationship to diagnostic certainty, the diagnostic accuracy and clinical utility of system 100 is higher than in prior systems.

System 100 utilizes the human brain's natural interpretation of a wide range of sound (and/or other sensory) patterns to localize abnormalities within the lung, thereby increasing the accuracy for specific diagnoses and their severity based upon the audio (and/or other sensory output) output from system 100. System 100 may have a wide range of applications as a simplified diagnostic technique useable by a wide range of new users, formerly unfamiliar with lung ultrasound. System 100 simplifies prior lung ultrasound techniques and can be used safely and effectively by primary healthcare providers, nurses, emergency personnel, caregivers, subjects themselves, etc.

In some embodiments, system 100 provides a new diagnostic model for use by patients themselves under the supervision of a physician. Such supervision can occur using telehealth methods.

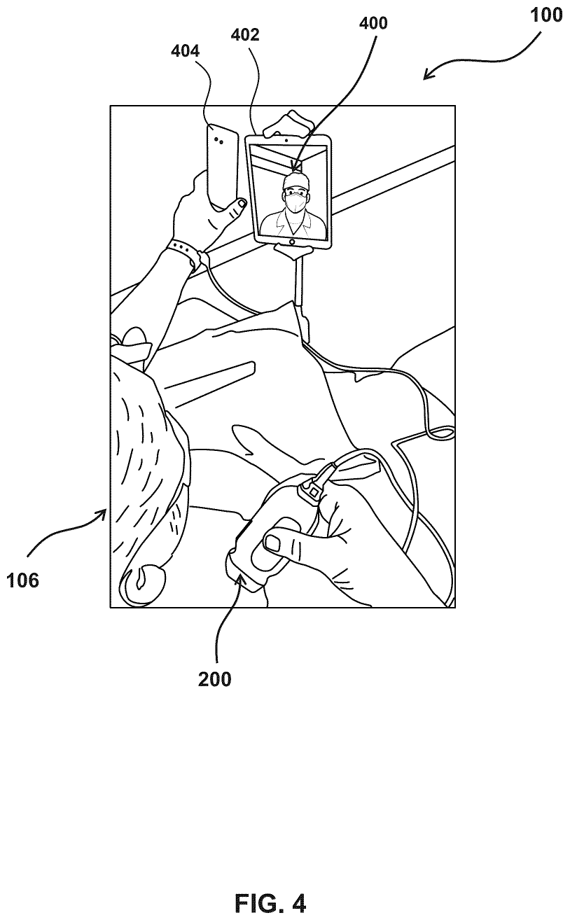

For example, FIG. 4 illustrates a subject 106 using system 100 (including an ultrasound sensor 200 in this example—and a sensory output device 202, one or more processors 204 and/or other components of system 100 not shown in FIG. 4) to detect areas of lung thickening. Subject 106 is showing results to a physician 400 using telehealth methods (e.g., by way of a tablet 402 and the subject's smartphone 404). In this example, physician 400 may also be able to hear (or see depending on the nature of sensory output from sensory output device 202) an audible indication (e.g., an increasingly frequent and/or loud) of lung thickening along with subject 106 as system 100 is moved about the chest of subject 106. Visual output may also be used (e.g., simultaneously or otherwise). This is an example of the device's use during a telehealth appointment with a healthcare provider, as previously described. For COVID-19 as one example, the patient may send this data from isolation. In other cases, using a visual output (see below, “stacked circles”), the physician can recognize and locate the abnormality using the camera. During pure audio output, the physician or patient can localize the abnormal region by sound alone. In some embodiments, system 100 may output simultaneous audio and, if connected to a screen, visual output (again see stacked circles below).

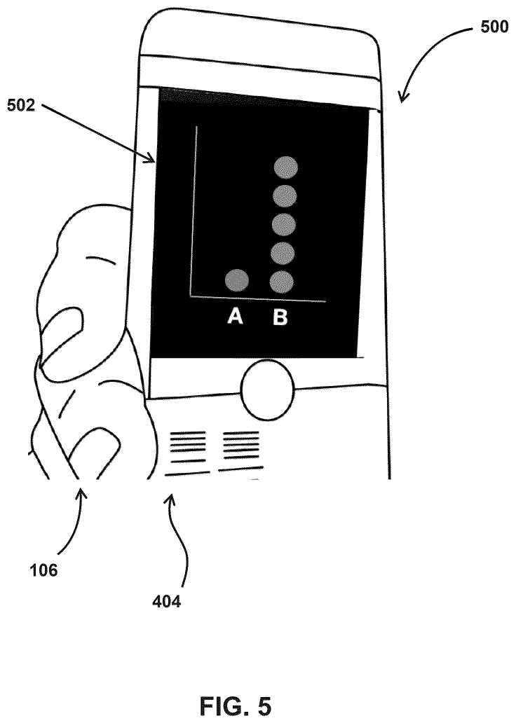

FIG. 5 illustrates an example of what subject 106 may present visually to physician 400 (FIG. 4) via the subject's smartphone 404. Subject 106 may present a graphical and/or other representation depicting a severity and/or other characteristics related to A-lines, B-lines, and/or other indications of lung thickening. As one example, the subject may present an ultrasound image (or video) comprising B-lines, A-lines, and/or other features. System 100 (FIGS. 1A, 2) is configured (as described above) to recognize these B-lines (and/or information in the output signals from sensor 200 that is indicative of B-lines) and generate sensory output.

As another example, as shown in FIG. 5, the presence of B-lines may be used to create a stacked graph 500 of colored circles 502 that displays the number of B-lines (1, 2, 3, 4, or more). It may also show A-lines, for example, as different colored circles. The height of the stacked circles, its color, and/or other properties, may be used to determine a severity of a finding. In addition, subject 106 may remember where system 100 (or the ultrasound portion thereof) is located by simultaneous audio output.

In some embodiments, smartphone 404 visual output displaying a bar graph with circles of A-lines and B-lines may be continually updated while moving system 100. This may also be accompanied by audio clicks, for example. Subject 106 may hold smartphone 404 to a camera (e.g., of a tablet computer) during a telehealth appointment (e.g., as described in FIG. 4).

These examples of graphical representations should not be considered limiting. Other examples of graphical representations are contemplated. System 100 may be configured such that subject 106 may present any graphical and/or other representation depicting a severity and/or other characteristics related to A-lines, B-lines, and/or other indications of lung thickening.

Returning to FIG. 2, one or more computing devices 206 may be and/or include a smartphone, a laptop computer, a tablet, a desktop computer, a gaming device, and/or other networked computing devices, having a display, a user input device (e.g., buttons, keys, voice recognition, or a single or multi-touch touchscreen), memory (such as a tangible, machine-readable, non-transitory memory), a network interface, an energy source (e.g., a battery), and a processor such as a processor 204 (a term which, as used herein, includes one or more processors) coupled to each of these components. Memory such as electronic storage 238 of computing device 206 may store instructions that when executed by the associated processor provide an operating system and various applications, including a web browser or a native mobile application, for example. In addition, computing device 206 may include a user interface 236, which may include a monitor; a keyboard; a mouse; a touchscreen; etc. User interface 236 may be operative to provide a graphical user interface associated with the system 100 that communicates with sensor 200, sensory output device 202, and/or processor(s) 204, and facilitates user interaction with data from sensor 200.

User interface 236 is configured to provide an interface between system 100 and users (e.g., subject 106 shown in FIG. 1A, a physician, etc.) through which users may provide information to and receive information from system 100. This enables data, results, and/or instructions, and any other communicable items, collectively referred to as “information,” to be communicated between the users and one or more of sensor 200, sensory output device 202, processor(s) 204, computing device 206, external resources 208, and/or other components. Examples of interface devices suitable for inclusion in user interface 236 include a keypad, buttons, switches, a keyboard, knobs, levers, a display screen, a touch screen, speakers, a microphone, an indicator light, an audible alarm, a printer, and/or other interface devices. In one embodiment, user interface 236 includes a plurality of separate interfaces (e.g., an interface on sensor 200, an interface on sensory output device 202, an interface in computing device 206, etc.). In one embodiment, user interface 236 includes at least one interface that is provided integrally with processor(s) 204. It is to be understood that many communication techniques, either hard-wired or wireless, between one or more components of system 100 are contemplated by the present disclosure. Other exemplary input devices and techniques adapted for use with system 100 as user interface 236 include, but are not limited to, an RS-232 port, RF link, an IR link, modem (telephone, cable or other). In short, any technique for communicating information with system 100 is contemplated by the present disclosure as user interface 236.

Electronic storage 238 comprises electronic storage media that electronically stores information. The electronic storage media of electronic storage 238 may include one or both of system storage that is provided integrally (i.e., substantially non-removable) with system 100 and/or removable storage that is removably connectable to system 100 via, for example, a port (e.g., a USB port, a firewire port, etc.) or a drive (e.g., a disk drive, etc.). Electronic storage 238 may include one or more of optically readable storage media (e.g., optical disks, etc.), magnetically readable storage media (e.g., magnetic tape, magnetic hard drive, floppy drive, etc.), electrical charge-based storage media (e.g., EEPROM, RAM, etc.), solid-state storage media (e.g., flash drive, etc.), and/or other electronically readable storage media. Electronic storage 238 may store software algorithms, information determined by processor(s) 204, information received via user interface 236, and/or other information that enables system 100 to function properly. Electronic storage 238 may be (in whole or in part) a separate component within system 100, or electronic storage 238 may be provided (in whole or in part) integrally with one or more other components of system 100 (e.g., computing device 206, processor(s) 204, etc.).

External resources 208, in some embodiments, include sources of information such as databases, websites, etc.; external entities participating with system 100 (e.g., systems or networks associated with system 100), one or more servers outside of the system 100, a network (e.g., the internet), electronic storage, equipment related to Wi-Fi™ technology, equipment related to Bluetooth® technology, data entry devices, or other resources. In some implementations, some or all of the functionality attributed herein to external resources 208 may be provided by resources included in system 100. External resources 208 may be configured to communicate with one or more other components of system 100 via wired and/or wireless connections, via a network (e.g., a local area network and/or the internet), via cellular technology, via Wi-Fi technology, and/or via other resources.

Network 250 may include the internet, a Wi-Fi network, Bluetooth® technology, and/or other wireless technology. In some embodiments, sensor 200, sensory output device 202, one or more processors 204, computing device 206, external resources 208, and/or other components of system 100 communicate via near field communication, Bluetooth, and/or radio frequency; via network 250 (e.g., a network such as a Wi-Fi network, a cellular network, and/or the internet); and/or by other communication methods.

In FIG. 2, sensor 200, sensory output device 202, one or more processors 204, one or more computing devices 206, and/or other components of system 100 are shown as separate entities. This is not intended to be limiting. Some and/or all of the components of system 100 and/or other components may be grouped into one or more singular devices. For example, sensor 200, sensory output device 202, and one or more processors 204 may be included in a computing device 206. These and/or other components may be included in a wearable worn by the subject 12. The wearable may be a garment, a device, and/or other wearables. Such a wearable may include means to deliver sensory output (e.g., a wired and/or wireless audio device and/or other devices) such as one or more audio speakers. The wearable device may be configured to be worn at or near an area of lung thickening, for example. In some embodiments, the wearable device may be and/or include a necklace, a chest strap, a shirt, a vest, a self-adherent transducer patch placed on the skin over the chest region, a watch or a watch band coupled to system 100 configured to transiently apply system 100 to the chest, and/or any other wearable device configured such that sensor 200, sensory output device 202, one or more processors 204, and/or other components of system 100 are positioned at or near an area of lung thickening.

In addition, in some embodiments, system 100 may include an ultrasound apparatus that is simplified and less costly to produce compared to prior apparatuses, because only an output signal that can be converted to sensory output may be needed. This type of device may lend itself to inclusion in wearables such as watches, pendant necklaces or garments embedded with ultrasound capability that could make sounds or change color (as two examples) based upon detection of lung thickening.

For example, in some embodiments, system 100 may be used by a user to determine where an abnormality is located (as indicated by clicks or other sensory output described above). A wearable may then be worn at or near that spot to assess progression or resolution, intermittently. In that way, a user is not required to keep searching for thickened lung areas with each use of system 100. This may be thought of as a method to “mark” the chest wall for the location of the abnormality (-ies). In other words, a memorable signal (e.g., audio clicks) may be used to “map” a lung thickening epicenter along with edges of the abnormality, so that one can return easily to the area in the future. Currently, this epicenter is found by the laborious counting of each individual B-line, which is not as memorable as the audio (or light) search for a “target.”

In some embodiments, system 100 (e.g., comprising a sensor 200, a sensory output device 202, one or more processors 204, etc. shown in FIG. 1 and FIG. 2) may be configured such that sensor 200 is configured to move proximate to a target area in an organ (e.g., a lung or some other organ such as a heart, liver, etc.) of subject 106, and generate one or more output signals comprising information indicating (1) a location of sensor 200 relative to the target area, and (2) presence of an abnormality of interest (e.g., a B line as described above, or some other abnormality of interest appropriate for a given organ) at or near the target area. Sensory output device 202 is configured to generate sensory output indicating (1) the location of sensor 200 relative to the target area, and (2) the presence of the abnormality of interest at or near the target area as sensor 200 moves proximate to the organ. One or more processors 204 are configured by machine readable instructions, to execute a trained machine learning model (e.g., as described above) to (1) determine the location of sensor 200 relative to the target area, and (2) detect the presence of the abnormality of interest, based on the output signals. One or more processors 204 are configured to control sensory output device 202 to generate and/or modulate the sensory output at or near the organ and/or the target area when sensor 200 moves proximate to the organ and/or the target area. In some embodiments, the generating and/or modulating comprises causing the sensory output to modulate as sensor 200 is moved nearer to, or further from, the organ and/or the target area, such that the sensory output indicates a location of the target area to subject 106. In some embodiments, the generating and/or modulating comprises, responsive to sensor 200 being located proximate to the organ and/or the target area (e.g., by subject 106 and/or some other user), causing the sensory output to indicate spatially localized areas of the target area with the abnormality of interest when the sensor moves around the target area.

In some embodiments, one or more processors 204 are configured to execute machine readable instructions (e.g., as described herein) for determining the location of the sensor relative to the target area, detecting the presence of the abnormality of interest, controlling the sensory output device to generate and/or modulate the sensory output, and/or other operations. The machine readable instructions may be configured to be changeable based on the organ (e.g., a lung, the heart, the liver, etc.), the target area (e.g., a specific anatomical feature of an organ), the abnormality of interest (e.g., a B line in a lung, a malfunctioning valve in a heart, etc.), and/or other factors, for example.

In some embodiments, one or more processors 204 may be configured to use pre-image signals from sensor 200 itself for determining the location of the sensor relative to the target area, detecting the presence of the abnormality of interest, controlling the sensory output device to generate and/or modulate the sensory output, and/or other operations. One or more processors 204 may be configured to use pre-image (e.g., radiofrequency) signals from sensor 200 (e.g., an ultrasound transducer) itself before any image processing occurs, so that image processing, a display screen, and/or other components are not necessary to include in system 100. This may greatly reduce the system's potential size—since it would be screenless and free of controls needed to generate, manipulate, adjust, etc., images.

By way of a non-limiting example, sound assignments localize pathology (e.g., a specific organ and/or a target area in an organ), as exemplified by the cluttering of sounds of a Geiger counter or squeal of a metal detector, with sites being remembered by subject 106 more readily than when displayed by numerical or visual outputs (for example). The ease with which a sound algorithm can be programmed and uploaded underscores the potential of a small device (e.g., system 100) to emit tones (and/or provide other sensory output) updated to guide sensor 200 (e.g., an ultrasound transducer) location to a specific region for diagnosis and/or other purposes. System 100 may be applied daily at a determined location, e.g., while performing exercise or at a time of dyspnea, etc., to listen for inducible interstitial edema or during upper respiratory infections to listen for progression to pneumonitis (as non-limiting examples).

Some prior devices use sound or tactile output as simply an alarm. These devices may be used for liver and fetal ultrasound, as examples—not the lung and/or other organs, where findings require more interpretative finesse, and therefore more sophisticated audio output.

These prior devices use sound or tactile output as simply an alarm from a wearable watch (or similar) when the received ultrasound signal does not match a normal baseline. In contrast, system 100 (e.g., including a diagnostic device itself such as an ultrasound) to emit sounds as it finds an abnormality during simultaneous imaging (e.g., with the computing components described herein made small enough for an audio-device only, without uploading to a phone, or used in combination with a phone and/or other components as described herein).

System 100 provides value in the sound algorithm used to display ultrasound findings. It is not simply an alarm when a threshold is exceeded, but an actual audio representation/display of a finding within the ultrasound signal. In system 100, there is a relationship with severity which allows localization and optimization of the probe position by the user. The system 100 AI sound output algorithm(s) may be configured based on expert interpretation and clinical opinion of the finding severity, to utilize the wide discriminatory ability of human hearing to understand the clinical value of the ultrasound finding(s). Many prior devices use a “catastrophic” one-dimensional threshold value in order to simply trigger an “alarm” to the user, or 911. System 100 interprets a spectrum, can use AI to “manipulate” its thresholds to produce various sounds, and/or utilize characteristics of human hearing for final decision-making (i.e., is an abnormality worse than before? is an abnormality moving and involving more areas?, etc.).

Sometimes, prior devices simply assign “volume” of their output to the differences in the ultrasound values compared to a normal baseline. System 100 may be configured to manipulate the audio output (e.g., tone, pitch, duration, tempo, timber, frequency, sequencing, etc.) to affect user interpretation of an abnormality finding in regard to its severity. As a simple example, faint B-lines may cause a “soft” sound, or multiple bright B-lines will cause a “louder” or “higher pitch” sound. This ability to assign specific audible signals related to the interpretation of the ultrasound data and its clinical value is different than simply assigning an alarm to a threshold difference.

Output from system 100 is not simply “present/absent” as an alarm. Prior devices may be likened to a metal detector that simply alarms a watch when there is metal somewhere on a beach. As clinical value or image severity is not taken into account, a typical prior device cannot localize the position of a worst finding. Prior devices often require precise placement of a transducer element (e.g., likely over the fetus or liver, continuing with the example above) and optimization of the signal, a priori. It cannot move and therefore is applicable for “ultrasound patch” placement, but not the varied applications of system 100. System 100 can “follow” and “search” for abnormalities around the body by leveraging the natural capability of human hearing, including specialized echoic memory, rapid temporal discrimination and binaural localization.

In regard to guiding transducer location, prior devices do not use sound to help find a correct position for imaging. Again, prior devices do not use ultrasound data (as one example) to help optimize for detecting pathologic findings. System 100 causes the user to move a probe to get the most informative audio output.

The illustrated components of system 100 are depicted as discrete functional blocks, but embodiments are not limited to systems in which the functionality described herein is organized as illustrated by FIG. 2. The functionality provided by each of the components of system 100 may be provided by software or hardware modules that are differently organized than is presently depicted, for example such software or hardware may be intermingled, broken up, distributed (e.g. within a data center or geographically), or otherwise differently organized. Some or all of the functionality described herein may be provided by one or more processors of one or more computers executing code stored on a tangible, non-transitory, machine readable medium.

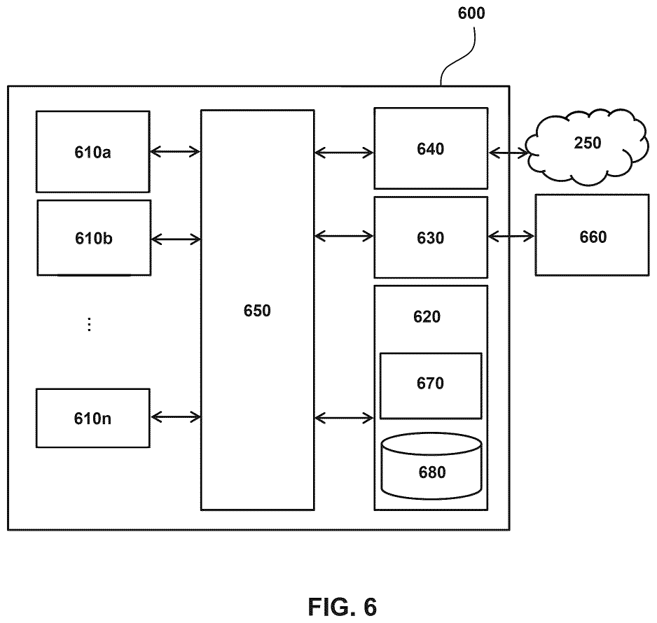

FIG. 6 is a diagram that illustrates an exemplary computing device 600 (similar to and/or the same as computing device 206 described above) in accordance with embodiments of the present system. Various portions of systems and methods described herein, may include, or be executed on one or more computing devices the same as or similar to computing device 600. For example, processor(s) 204 of system 100 (FIG. 2) may be and/or be included in one more computing devices the same as or similar to computing device 600. Further, processes, modules, processor components, and/or other components of system 100 described herein may be executed by one or more processing systems similar to and/or the same as that of computing device 600.

Computing device 600 may include one or more processors (e.g., processors 610a-610n, which may be similar to and or the same as processor(s) 204) coupled to system memory 620 (which may be similar to and/or the same as electronic storage 238, an input/output I/O device interface 630, and a network interface 640 via an input/output (I/O) interface 650. A processor may include a single processor or a plurality of processors (e.g., distributed processors). A processor may be any suitable processor capable of executing or otherwise performing instructions. A processor may include a central processing unit (CPU) that carries out program instructions to perform the arithmetical, logical, and input/output operations of computing device 600. A processor may execute code (e.g., processor firmware, a protocol stack, a database management system, an operating system, or a combination thereof) that creates an execution environment for program instructions. A processor may include a programmable processor. A processor may include general or special purpose microprocessors. A processor may receive instructions and data from a memory (e.g., system memory 620). Computing device 600 may be a uni-processor system including one processor (e.g., processor 610a), or a multi-processor system including any number of suitable processors (e.g., 610a-610n). Multiple processors may be employed to provide for parallel or sequential execution of one or more portions of the techniques described herein. Processes, such as logic flows, described herein may be performed by one or more programmable processors executing one or more computer programs to perform functions by operating on input data and generating corresponding output. Processes described herein may be performed by, and apparatus can also be implemented as, special purpose logic circuitry, e.g., an FPGA (field programmable gate array) or an ASIC (application specific integrated circuit). Computing device 600 may include a plurality of computing devices (e.g., distributed computer systems) to implement various processing functions.

I/O device interface 630 may provide an interface for connection of one or more I/O devices 660 to computer device 600. I/O devices may include devices that receive input (e.g., from a user) or output information (e.g., to a user). I/O devices 660 may include, for example, graphical user interface presented on displays (e.g., a cathode ray tube (CRT) or liquid crystal display (LCD) monitor), pointing devices (e.g., a computer mouse or trackball), keyboards, keypads, touchpads, scanning devices, voice recognition devices, gesture recognition devices, printers, audio speakers, microphones, cameras, or the like. I/O devices 660 may be connected to computing device 600 through a wired or wireless connection. I/O devices 660 may be connected to computing device 600 from a remote location. I/O devices 660 located on remote computer system, for example, may be connected to computing device 600 via a network and network interface 640.

Network interface 640 may include a network adapter that provides for connection of computing device 600 to a network (e.g., network 250 described above). Network interface may 640 may facilitate data exchange between computing device 600 and other devices connected to the network (e.g., network 250 shown in FIG. 2). Network interface 640 may support wired or wireless communication. The network may include an electronic communication network, such as the Internet, a local area network (LAN), a wide area network (WAN), a cellular communications network, or the like.

System memory 620 may be configured to store program instructions 670 (e.g., machine readable instructions) or data 680. Program instructions 670 may be executable by a processor (e.g., one or more of processors 610a-610n) to implement one or more embodiments of the present techniques. Instructions 670 may include modules and/or components of computer program instructions for implementing one or more techniques described herein with regard to various processing modules and/or components. Program instructions may include a computer program (which in certain forms is known as a program, software, software application, script, or code). A computer program may be written in a programming language, including compiled or interpreted languages, or declarative or procedural languages. A computer program may include a unit suitable for use in a computing environment, including as a stand-alone program, a module, a component, or a subroutine. A computer program may or may not correspond to a file in a file system. A program may be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program may be deployed to be executed on one or more computer processors located locally at one site or distributed across multiple remote sites and interconnected by a communication network.

System memory 620 may include a tangible program carrier having program instructions stored thereon. A tangible program carrier may include a non-transitory computer readable storage medium. A non-transitory computer readable storage medium may include a machine readable storage device, a machine readable storage substrate, a memory device, or any combination thereof. Non-transitory computer readable storage medium may include non-volatile memory (e.g., flash memory, ROM, PROM, EPROM, EEPROM memory), volatile memory (e.g., random access memory (RAM), static random access memory (SRAM), synchronous dynamic RAM (SDRAM)), bulk storage memory (e.g., CD-ROM and/or DVD-ROM, hard-drives), or the like. System memory 620 may include a non-transitory computer readable storage medium that may have program instructions stored thereon that are executable by a computer processor (e.g., one or more of processors 610a-610n) to cause the subject matter and the functional operations described herein. A memory (e.g., system memory 620) may include a single memory device and/or a plurality of memory devices (e.g., distributed memory devices). Instructions or other program code to provide the functionality described herein may be stored on a tangible, non-transitory computer readable media. In some cases, the entire set of instructions may be stored concurrently on the media, or in some cases, different parts of the instructions may be stored on the same media at different times, e.g., a copy may be created by writing program code to a first-in-first-out buffer in a network interface, where some of the instructions are pushed out of the buffer before other portions of the instructions are written to the buffer, with all of the instructions residing in memory on the buffer, just not all at the same time.

I/O interface 650 may be configured to coordinate I/O traffic between processors 610a-610n, system memory 620, network interface 640, I/O devices 660, and/or other peripheral devices. I/O interface 650 may perform protocol, timing, or other data transformations to convert data signals from one component (e.g., system memory 620) into a format suitable for use by another component (e.g., processors 610a-610n). I/O interface 650 may include support for devices attached through various types of peripheral buses, such as a variant of the Peripheral Component Interconnect (PCI) bus standard or the Universal Serial Bus (USB) standard.

Embodiments of the techniques described herein may be implemented using a single instance of computing device 600 or multiple computing devices 600 configured to host different portions or instances of embodiments. Multiple computing devices 600 may provide for parallel or sequential processing/execution of one or more portions of the techniques described herein.

Those skilled in the art will appreciate that computing device 600 is merely illustrative and is not intended to limit the scope of the techniques described herein. Computing device 600 may include any combination of devices or software that may perform or otherwise provide for the performance of the techniques described herein. For example, computing device 600 may include or be a combination of a cloud-computing system, a data center, a server rack, a server, a virtual server, a desktop computer, a laptop computer, a tablet computer, a server device, a client device, a mobile telephone, s smartphone, a personal digital assistant (PDA), a mobile audio or video player, a game console, a vehicle-mounted computer, or a Global Positioning System (GPS), or the like. Computing device 600 may also be connected to other devices that are not illustrated, or may operate as a stand-alone system. In addition, the functionality provided by the illustrated components may in some embodiments be combined in fewer components or distributed in additional components. Similarly, in some embodiments, the functionality of some of the illustrated components may not be provided or other additional functionality may be available.

Those skilled in the art will also appreciate that while various items are illustrated as being stored in memory or on storage while being used, these items or portions of them may be transferred between memory and other storage devices for purposes of memory management and data integrity. Alternatively, in other embodiments some or all of the software components may execute in memory on another device and communicate with the illustrated computer system via inter-computer communication. Some or all of the system components or data structures may also be stored (e.g., as instructions or structured data) on a computer-accessible medium or a portable article to be read by an appropriate drive, various examples of which are described above. In some embodiments, instructions stored on a computer-accessible medium separate from computing device 600 may be transmitted to computing device 600 via transmission media or signals such as electrical, electromagnetic, or digital signals, conveyed via a communication medium such as a network or a wireless link. Various embodiments may further include receiving, sending, or storing instructions or data implemented in accordance with the foregoing description upon a computer-accessible medium. Accordingly, the present invention may be practiced with other computer system configurations.

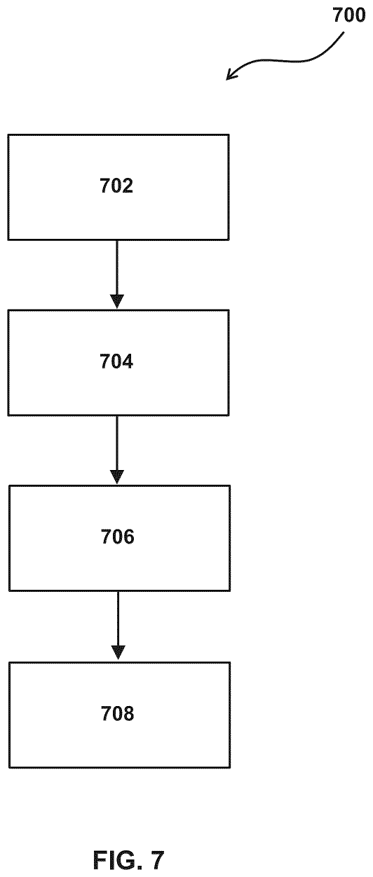

FIG. 7 illustrates a method 700 for providing sensory output indicating lung thickening in a subject. The operations of method 700 presented below are intended to be illustrative. In some embodiments, method 700 may be accomplished with one or more additional operations not described, and/or without one or more of the operations discussed. Additionally, the order in which the operations of method 700 are illustrated in FIG. 7 and described below is not intended to be limiting.

In some embodiments, some or all of method 700 may be implemented in one or more processing devices (e.g., a digital processor, an analog processor, a digital circuit designed to process information, an analog circuit designed to process information, a state machine, and/or other mechanisms for electronically processing information). The one or more processing devices (e.g., processor(s) 204, processor 610a, etc., described herein) may include one or more devices executing some or all of the operations of method 700 in response to instructions stored electronically on an electronic storage medium (e.g., electronic storage 238, system memory 620, etc.). The one or more processing devices may include one or more devices configured through hardware, firmware, and/or software to be specifically designed for execution of one or more of the operations of method 700.

At an operation 702, a sensor is moved proximate to one or both lungs of the subject. The sensor is configured to be moved by the subject and/or other users. The sensor movement proximate to one or both lungs of the subject comprises a back and forth rastering motion across the lungs and/or other movement. The sensor is configured to generate one or more output signals comprising information indicating areas of lung thickening due to congestion, infection, inflammation, and/or fibrosis. In some embodiments, the sensor comprises an ultrasound apparatus. The one or more output signals may comprise, and/or may be used to generate, raw radiofrequency data, one or more images of the lungs of the subject, pre and post processed scan lines, a constructed postprocessed 2D image, and/or other information. In some embodiments, the one or more output signals indicate B-lines associated with the lungs of the subject. In some embodiments, operation 702 is performed by or with a sensor similar to and/or the same as sensor 200 (shown in FIG. 2 and described herein).

At an operation 704, sensory output indicating the areas of lung thickening is generated with a sensory output device as the sensor moves proximate to the lungs. The sensory output is configured to facilitate detection and localization of abnormalities associated with lung thickening in the lungs. The sensory output comprises: auditory output including one or more sounds generated by the sensory output device; haptic output including one or more vibrations or patterns of vibrations generated by the sensory output device; visual output including one or more flashes or patterns of flashes of light, and/or one or more colors or patterns of colors of light, generated by the sensory output device; and/or other sensory output. In some embodiments, the sensory output intensifies or otherwise modulates as the sensor moves closer to an area of lung thickening. In some embodiments, the sensory output comprises auditory output, and the auditory output comprises a series of clicks configured to intensify as the sensor moves closer to an area of lung thickening. Intensification may comprise an increase in click volume and/or click frequency, for example. In some embodiments, operation 704 is performed by a sensory output device the same as or similar to sensory output device 202 (shown in FIG. 2 and described herein).

At an operation 706, a trained machine learning model is executed, with one or more processors configured by machine readable instructions, to detect the areas of lung thickening based on the output signals. The machine learning model may comprise a deep neural network, for example. In some embodiments, the trained machine learning model is trained by obtaining and providing prior sensor output signals associated with areas of lung thickening to the machine learning model. Portions of the prior sensor output signals associated with the areas of lung thickening may be labeled as thickened lung areas. In some embodiments, the trained machine learning model is trained with training data. The training data may comprise input output training pairs comprising a labeled B-line in an output signal and a corresponding indication of spatially localized area of the lungs. In some embodiments, the trained machine learning model is trained to detect the areas of lung thickening based on the B-lines. In some embodiments, the trained machine learning model is trained based on database ultrasound images that have been ranked according to B-line number and echogenicity, representing diagnostic certainty for an abnormal image such that the more numerous and distinct the B-lines appear on an image the more recognizable the sensory output. The database ultrasound images comprise extracted individual frames from a video, where an annotation tool has been applied to visualize and/or label relevant properties of a frame, and where, for image classification, relevant properties include an estimated severity score, an annotator's confidence score for the estimated severity score, a number of B-lines, and/or a number of A-lines. The trained machine learning model is configured to use temporal characteristics of training data including disappearance of B-lines during respiration and data averaging to assess severity of lung thickening, so as to avoid errors due to evanescence or confluence of B-lines. The trained machine learning model is trained on a dataset that is uniquely interpreted cognizant to a finding's relationship to diagnostic certainty, such that a diagnostic accuracy and clinical utility of the system higher than prior systems. In some embodiments, operation 706 is performed by a processor the same as or similar to processor(s) 204 (shown in FIG. 2 and described herein).

At an operation 708, the sensory output device is controlled, with the one or more processors, to generate and/or modulate the sensory output at spatially localized areas of lung thickening when the sensor moves proximate to the areas of lung thickening. In some embodiments, detecting the areas of lung thickening based on the output signals, and controlling the sensory output device to generate and/or modulate the sensory output at the areas of lung thickening, occurs in real time as the sensor moves proximate to the areas of lung thickening in the subject. In some embodiments, operation 708 is performed by a processor the same as or similar to processor(s) 204 (shown in FIG. 2 and described herein.)

In block diagrams, illustrated components are depicted as discrete functional blocks, but embodiments are not limited to systems in which the functionality described herein is organized as illustrated. The functionality provided by each of the components may be provided by software or hardware modules that are differently organized than is presently depicted, for example such software or hardware may be intermingled, conjoined, replicated, broken up, distributed (e.g. within a data center or geographically), or otherwise differently organized. The functionality described herein may be provided by one or more processors of one or more computers executing code stored on a tangible, non-transitory, machine readable medium. In some cases, notwithstanding use of the singular term “medium,” the instructions may be distributed on different storage devices associated with different computing devices, for instance, with each computing device having a different subset of the instructions, an implementation consistent with usage of the singular term “medium” herein. In some cases, third party content delivery networks may host some or all of the information conveyed over networks, in which case, to the extent information (e.g., content) is said to be supplied or otherwise provided, the information may provided by sending instructions to retrieve that information from a content delivery network.

Various embodiments of the present systems and methods are disclosed in the subsequent list of numbered clauses. In the following, further features, characteristics, and exemplary technical solutions of the present disclosure will be described in terms of clauses that may be optionally claimed in any combination:
1. A system configured to provide sensory output indicating lung thickening in a subject, the system comprising: a sensor configured to move proximate to one or both lungs of the subject, the sensor configured to generate one or more output signals comprising information indicating areas of lung thickening due to congestion, infection, inflammation, and/or fibrosis; a sensory output device configured to generate sensory output indicating the areas of lung thickening as the sensor moves proximate to the lungs; and one or more processors configured by machine readable instructions to: execute a trained machine learning model to detect the areas of lung thickening based on the output signals; and control the sensory output device to generate and/or modulate the sensory output at spatially localized areas of lung thickening when the sensor moves proximate to the areas of lung thickening.
2. The system of clause 1, wherein the sensor comprises an ultrasound apparatus.
3. The system of any of the previous clauses, where detecting the areas of lung thickening based on the output signals, and controlling the sensory output device to generate and/or modulate the sensory output at the areas of lung thickening, occurs in real time as the sensor moves proximate to the areas of lung thickening in the subject.
4. The system of any of the previous clauses, wherein sensor movement proximate to one or both lungs of the subject comprises a back and forth rastering motion across the lungs.
5. The system of any of the previous clauses, wherein the sensory output comprises: auditory output including one or more sounds generated by the sensory output device; haptic output including one or more vibrations or patterns of vibrations generated by the sensory output device;
and/or visual output including one or more flashes or patterns of flashes of light, and/or one or more colors or patterns of colors of light, generated by the sensory output device.
6. The system of any of the previous clauses, wherein the sensory output intensifies or otherwise modulates as the sensor moves closer to an area of lung thickening.
7. The system of any of the previous clauses, wherein the sensory output comprises auditory output, and the auditory output comprises a series of clicks configured to intensify as the sensor moves closer to an area of lung thickening.
8. The system of any of the previous clauses, wherein intensification comprises an increase in click volume and/or click frequency.
9. The system of any of the previous clauses, wherein the one or more output signals comprise, and/or are used to generate, raw radiofrequency data, one or more images of the lungs of the subject, pre and post processed scan lines, and/or a constructed postprocessed 2D image.
10. The system of any of the previous clauses, wherein the one or more output signals indicate B-lines associated with the lungs of the subject, and wherein the trained machine learning model is trained to detect the areas of lung thickening based on the B-lines.
11. The system of any of the previous clauses, wherein the trained machine learning model is trained by: obtaining and providing prior sensor output signals associated with areas of lung thickening to the machine learning model; wherein portions of the prior sensor output signals associated with the areas of lung thickening are labeled as thickened lung areas.
12. The system of any of the previous clauses, wherein the trained machine learning model is trained with training data, the training data comprising input output training pairs comprising a labeled B-line in an output signal and a corresponding indication of spatially localized area of the lungs.
13. The system of claim 1, wherein the sensory output is configured to facilitate detection and localization of abnormalities associated with lung thickening in the lungs.
14. The system of any of the previous clauses, wherein the trained machine learning model is trained based on database ultrasound images that have been ranked according to B-line number and echogenicity, representing diagnostic certainty for an abnormal image such that the more numerous and distinct the B-lines appear on an image the more recognizable the sensory output.
15. The system of any of the previous clauses, wherein the database ultrasound images comprise extracted individual frames from a video, where an annotation tool has been applied to visualize and/or label relevant properties of a frame, and where, for image classification, relevant properties include an estimated severity score, an annotator's confidence score for the estimated severity score, a number of B-lines, and/or a number of A-lines.
16. The system of any of the previous clauses, wherein the trained machine learning model is configured to use temporal characteristics of training data including disappearance of B-lines during respiration and data averaging to assess severity of lung thickening, so as to avoid errors due to evanescence or confluence of B-lines.
17. The system of any of the previous clauses, wherein the trained machine learning model is trained on a dataset that is uniquely interpreted cognizant to a finding's relationship to diagnostic certainty, such that a diagnostic accuracy and clinical utility of the system is higher than prior systems.
18. The system of any of the previous clauses, wherein the sensor, the sensory output device, and the one or more processors comprise a wearable device, and wherein the wearable device is configured to be worn at or near an area of lung thickening.
19. The system of any of the previous clauses, wherein the machine learning model comprises a deep neural network.
20. The system of any of the previous clauses, wherein the sensor is configured to be moved by the subject.
21. A method for providing sensory output indicating lung thickening in a subject, the method comprising: moving a sensor proximate to one or both lungs of the subject, the sensor configured to generate one or more output signals comprising information indicating areas of lung thickening due to congestion, infection, inflammation, and/or fibrosis; generating, with a sensory output device, sensory output indicating the areas of lung thickening as the sensor moves proximate to the lungs; executing, with one or more processors configured by machine readable instructions, a trained machine learning model to detect the areas of lung thickening based on the output signals; and controlling, with the one or more processors, the sensory output device to generate and/or modulate the sensory output at spatially localized areas of lung thickening when the sensor moves proximate to the areas of lung thickening.
22. The method of clause 21, wherein the sensor comprises an ultrasound apparatus.
23. The method of any of the previous clauses, wherein detecting the areas of lung thickening based on the output signals, and controlling the sensory output device to generate and/or modulate the sensory output at the areas of lung thickening, occurs in real time as the sensor moves proximate to the areas of lung thickening in the subject.
24. The method of any of the previous clauses, wherein sensor movement proximate to one or both lungs of the subject comprises a back and forth rastering motion across the lungs.
25. The method of any of the previous clauses, wherein the sensory output comprises: auditory output including one or more sounds generated by the sensory output device; haptic output including one or more vibrations or patterns of vibrations generated by the sensory output device; and/or visual output including one or more flashes or patterns of flashes of light, and/or one or more colors or patterns of colors of light, generated by the sensory output device.
26. The method of any of the previous clauses, wherein the sensory output intensifies or otherwise modulates as the sensor moves closer to an area of lung thickening.
27. The method of any of the previous clauses, wherein the sensory output comprises auditory output, and the auditory output comprises a series of clicks configured to intensify as the sensor moves closer to an area of lung thickening.
28. The method of any of the previous clauses, wherein intensification comprises an increase in click volume and/or click frequency.
29. The method of any of the previous clauses, wherein the one or more output signals comprise, and/or are used to generate, raw radiofrequency data, one or more images of the lungs of the subject, pre and post processed scan lines, and/or a constructed postprocessed 2D image.
30. The method of any of the previous clauses, wherein the one or more output signals indicate B-lines associated with the lungs of the subject, and wherein the trained machine learning model is trained to detect the areas of lung thickening based on the B-lines.
31. The method of any of the previous clauses, wherein the trained machine learning model is trained by: obtaining and providing prior sensor output signals associated with areas of lung thickening to the machine learning model; wherein portions of the prior sensor output signals associated with the areas of lung thickening are labeled as thickened lung areas.
32. The method of any of the previous clauses, wherein the trained machine learning model is trained with training data, the training data comprising input output training pairs comprising a labeled B-line in an output signal and a corresponding indication of spatially localized area of the lungs.
33. The method of any of the previous clauses, wherein the sensory output is configured to facilitate detection and localization of abnormalities associated with lung thickening in the lungs.
34. The method of any of the previous clauses, wherein the trained machine learning model is trained based on database ultrasound images that have been ranked according to B-line number and echogenicity, representing diagnostic certainty for an abnormal image such that the more numerous and distinct the B-lines appear on an image the more recognizable the sensory output.
35. The method of any of the previous clauses, wherein the database ultrasound images comprise extracted individual frames from a video, where an annotation tool has been applied to visualize and/or label relevant properties of a frame, and where, for image classification, relevant properties include an estimated severity score, an annotator's confidence score for the estimated severity score, a number of B-lines, and/or a number of A-lines.
36. The method of any of the previous clauses, wherein the trained machine learning model is configured to use temporal characteristics of training data including disappearance of B-lines during respiration and data averaging to assess severity of lung thickening, so as to avoid errors due to evanescence or confluence of B-lines.
37. The method of any of the previous clauses, wherein the trained machine learning model is trained on a dataset that is uniquely interpreted cognizant to a finding's relationship to diagnostic certainty, such that a diagnostic accuracy and clinical utility of the system is higher than prior systems.
38. The method of any of the previous clauses, wherein the sensor, the sensory output device, and the one or more processors comprise a wearable device, and wherein the wearable device is configured to be worn at or near an area of lung thickening.
39. The method of any of the previous clauses, wherein the machine learning model comprises a deep neural network.
40. The method of any of the previous clauses, wherein the sensor is configured to be moved by the subject.
41. A non-transitory computer readable medium having instructions thereon, the instructions when executed by a computer causing the computer to perform operations comprising: receiving, from a sensor that is moved proximate to one or both lungs of a subject, one or more output signals comprising information indicating areas of lung thickening due to congestion, infection, inflammation, and/or fibrosis; executing a trained machine learning model to detect the areas of lung thickening based on the output signals; and controlling a sensory output device to generate and/or modulate sensory output at spatially localized areas of lung thickening when the sensor moves proximate to the areas of lung thickening.
42. The medium of clause 41, wherein the sensor comprises an ultrasound apparatus.
43. The medium of any of the previous clauses, where detecting the areas of lung thickening based on the output signals, and controlling the sensory output device to generate and/or modulate the sensory output at the areas of lung thickening, occurs in real time as the sensor moves proximate to the areas of lung thickening in the subject.
44. The medium of any of the previous clauses, wherein sensor movement proximate to one or both lungs of the subject comprises a back and forth rastering motion across the lungs.
45. The medium of any of the previous clauses, wherein the sensory output comprises: auditory output including one or more sounds generated by the sensory output device; haptic output including one or more vibrations or patterns of vibrations generated by the sensory output device; and/or visual output including one or more flashes or patterns of flashes of light, and/or one or more colors or patterns of colors of light, generated by the sensory output device.
46. The medium of any of the previous clauses, wherein the sensory output intensifies or otherwise modulates as the sensor moves closer to an area of lung thickening.
47. The medium of any of the previous clauses, wherein the sensory output comprises auditory output, and the auditory output comprises a series of clicks configured to intensify as the sensor moves closer to an area of lung thickening.
48. The medium of any of the previous clauses, wherein intensification comprises an increase in click volume and/or click frequency.
49. The medium of any of the previous clauses, wherein the one or more output signals comprise, and/or are used to generate, raw radiofrequency data, one or more images of the lungs of the subject, pre and post processed scan lines, and/or a constructed postprocessed 2D image.
50. The medium of any of the previous clauses, wherein the one or more output signals indicate B-lines associated with the lungs of the subject, and wherein the trained machine learning model is trained to detect the areas of lung thickening based on the B-lines.
51. The medium of any of the previous clauses, wherein the trained machine learning model is trained by: obtaining and providing prior sensor output signals associated with areas of lung thickening to the machine learning model; wherein portions of the prior sensor output signals associated with the areas of lung thickening are labeled as thickened lung areas.
52. The medium of any of the previous clauses, wherein the trained machine learning model is trained with training data, the training data comprising input output training pairs comprising a labeled B-line in an output signal and a corresponding indication of spatially localized area of the lungs.
53. The medium of any of the previous clauses, wherein the sensory output is configured to facilitate detection and localization of abnormalities associated with lung thickening in the lungs.
54. The medium of any of the previous clauses, wherein the trained machine learning model is trained based on database ultrasound images that have been ranked according to B-line number and echogenicity, representing diagnostic certainty for an abnormal image such that the more numerous and distinct the B-lines appear on an image the more recognizable the sensory output.
55. The medium of any of the previous clauses, wherein the database ultrasound images comprise extracted individual frames from a video, where an annotation tool has been applied to visualize and/or label relevant properties of a frame, and where, for image classification, relevant properties include an estimated severity score, an annotator's confidence score for the estimated severity score, a number of B-lines, and/or a number of A-lines.
56. The medium of any of the previous clauses, wherein the trained machine learning model is configured to use temporal characteristics of training data including disappearance of B-lines during respiration and data averaging to assess severity of lung thickening, so as to avoid errors due to evanescence or confluence of B-lines.
57. The medium of any of the previous clauses, wherein the trained machine learning model is trained on a dataset that is uniquely interpreted cognizant to a finding's relationship to diagnostic certainty, such that a diagnostic accuracy and clinical utility of the system is higher than prior systems.
58. The medium of any of the previous clauses, wherein the sensor, the sensory output device, and the one or more processors comprise a wearable device, and wherein the wearable device is configured to be worn at or near an area of lung thickening.
59. The medium of any of the previous clauses, wherein the machine learning model comprises a deep neural network.
60. The medium of any of the previous clauses, wherein the sensor is configured to be moved by the subject.
61. A system configured to provide sensory output indicating an abnormality of interest in a target area in an organ of a subject, the system comprising: a sensor configured to move proximate to the target area in the organ of the subject, the sensor configured to generate one or more output signals comprising information indicating (1) a location of the sensor relative to the target area, and (2) presence of the abnormality of interest at or near the target area; a sensory output device configured to generate sensory output indicating (1) the location of the sensor relative to the target area, and (2) the presence of the abnormality of interest at or near the target area as the sensor moves proximate to the organ; and one or more processors configured by machine readable instructions to: execute a trained machine learning model to (1) determine the location of the sensor relative to the target area, and (2) detect the presence of the abnormality of interest, based on the output signals; and control the sensory output device to generate and/or modulate the sensory output at or near the organ and/or the target area when the sensor moves proximate to the organ and/or the target area, the generating and/or modulating comprising: causing the sensory output to modulate as the sensor is moved nearer to, or further from, the organ and/or the target area, such that the sensory output indicates a location of the target area to the subject; and responsive to the sensor being located proximate to the organ and/or the target area, causing the sensory output to indicate spatially localized areas of the target area with the abnormality of interest when the sensor moves around the target area.
62. The system of any of the previous clauses, wherein the one or more processors are configured to execute machine readable instructions for determining the location of the sensor relative to the target area, detecting the presence of the abnormality of interest, and controlling the sensory output device to generate and/or modulate the sensory output, the machine readable instructions configured to be changeable based on the organ, the target area, and/or the abnormality of interest.
63. The system of any of the previous clauses, wherein the organ comprises a lung, a heart, or a liver of the subject.
64. The system of any of the previous clauses, wherein the sensor comprises an ultrasound apparatus, and wherein the sensory output comprises: auditory output including one or more sounds generated by the sensory output device; haptic output including one or more vibrations or patterns of vibrations generated by the sensory output device; and/or visual output including one or more flashes or patterns of flashes of light, and/or one or more colors or patterns of colors of light, generated by the sensory output device.
65. The system of any of the previous clauses, wherein the sensory output intensifies or otherwise modulates as the sensor moves closer to the abnormality of interest based on AI modeling.
66. A method for providing sensory output indicating an abnormality of interest in a target area in an organ of a subject, the method comprising: generating, with a sensor configured to move proximate to the target area in the organ of the subject, one or more output signals comprising information indicating (1) a location of the sensor relative to the target area, and (2) presence of the abnormality of interest at or near the target area; generating, with a sensory output device, sensory output indicating (1) the location of the sensor relative to the target area, and (2) the presence of the abnormality of interest at or near the target area as the sensor moves proximate to the organ; executing, with one or more processors, a trained machine learning model to (1) determine the location of the sensor relative to the target area, and (2) detect the presence of the abnormality of interest, based on the output signals; and controlling, with the one or more processors, the sensory output device to generate and/or modulate the sensory output at or near the organ and/or the target area when the sensor moves proximate to the organ and/or the target area, the generating and/or modulating comprising: causing the sensory output to modulate as the sensor is moved nearer to, or further from, the organ and/or the target area, such that the sensory output indicates a location of the target area to the subject; and responsive to the sensor being located proximate to the organ and/or the target area, causing the sensory output to indicate spatially localized areas of the target area with the abnormality of interest when the sensor moves around the target area.
67. The method of any of the previous clauses, wherein the one or more processors are configured to execute machine readable instructions for determining the location of the sensor relative to the target area, detecting the presence of the abnormality of interest, and controlling the sensory output device to generate and/or modulate the sensory output, the machine readable instructions configured to be changeable based on the organ, the target area, and/or the abnormality of interest.
68. The method of any of the previous clauses, wherein the organ comprises a lung, a heart, or a liver of the subject.
69. The method of any of the previous clauses, wherein the sensor comprises an ultrasound apparatus, and wherein the sensory output comprises: auditory output including one or more sounds generated by the sensory output device; haptic output including one or more vibrations or patterns of vibrations generated by the sensory output device; and/or visual output including one or more flashes or patterns of flashes of light, and/or one or more colors or patterns of colors of light, generated by the sensory output device.
70. The method of any of the previous clauses, wherein the sensory output intensifies or otherwise modulates as the sensor moves closer to the abnormality of interest.
71. A non-transitory computer readable medium having instructions thereon, the instructions when executed by a computer causing the computer to perform operations comprising: receiving, from a sensor configured to move proximate to a target area in an organ of a subject, one or more output signals comprising information indicating (1) a location of the sensor relative to the target area, and (2) presence of an abnormality of interest at or near the target area; causing generation, with a sensory output device, of sensory output indicating (1) the location of the sensor relative to the target area, and (2) the presence of the abnormality of interest at or near the target area as the sensor moves proximate to the organ; executing, with one or more processors, a trained machine learning model to (1) determine the location of the sensor relative to the target area, and (2) detect the presence of the abnormality of interest, based on the output signals; and controlling, with the one or more processors, the sensory output device to generate and/or modulate the sensory output at or near the organ and/or the target area when the sensor moves proximate to the organ and/or the target area, the generating and/or modulating comprising: causing the sensory output to modulate as the sensor is moved nearer to, or further from, the organ and/or the target area, such that the sensory output indicates a location of the target area to the subject; and responsive to the sensor being located proximate to the organ and/or the target area, causing the sensory output to indicate spatially localized areas of the target area with the abnormality of interest when the sensor moves around the target area.
72. The medium of any of the previous clauses, wherein the instructions configured to be changeable based on the organ, the target area, and/or the abnormality of interest.
73. The medium of any of the previous clauses, wherein the organ comprises a lung, a heart, or a liver of the subject.
74. The medium of any of the previous clauses, wherein the sensor comprises an ultrasound apparatus, and wherein the sensory output comprises: auditory output including one or more sounds generated by the sensory output device; haptic output including one or more vibrations or patterns of vibrations generated by the sensory output device; and/or visual output including one or more flashes or patterns of flashes of light, and/or one or more colors or patterns of colors of light, generated by the sensory output device.
75. The medium of any of the previous clauses, wherein the sensory output intensifies or otherwise modulates as the sensor moves closer to the abnormality of interest.

The reader should appreciate that the present application describes several inventions. Rather than separating those inventions into multiple isolated patent applications, applicants have grouped these inventions into a single document because their related subject matter lends itself to economies in the application process. But the distinct advantages and aspects of such inventions should not be conflated. In some cases, embodiments address all of the deficiencies noted herein, but it should be understood that the inventions are independently useful, and some embodiments address only a subset of such problems or offer other, unmentioned benefits that will be apparent to those of skill in the art reviewing the present disclosure. Due to costs constraints, some inventions disclosed herein may not be presently claimed and may be claimed in later filings, such as continuation applications or by amending the present claims. Similarly, due to space constraints, neither the Abstract nor the Summary of the Invention sections of the present document should be taken as containing a comprehensive listing of all such inventions or all aspects of such inventions.

It should be understood that the description and the drawings are not intended to limit the invention to the particular form disclosed, but to the contrary, the intention is to cover all modifications, equivalents, and alternatives falling within the spirit and scope of the present invention as defined by the appended claims. Further modifications and alternative embodiments of various aspects of the invention will be apparent to those skilled in the art in view of this description. Accordingly, this description and the drawings are to be construed as illustrative only and are for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed or omitted, and certain features of the invention may be utilized independently, all as would be apparent to one skilled in the art after having the benefit of this description of the invention. Changes may be made in the elements described herein without departing from the spirit and scope of the invention as described in the following claims. Headings used herein are for organizational purposes only and are not meant to be used to limit the scope of the description.

As used throughout this application, the word “may” is used in a permissive sense (i.e., meaning having the potential to), rather than the mandatory sense (i.e., meaning must). The words “include”, “including”, and “includes” and the like mean including, but not limited to. As used throughout this application, the singular forms “a,” “an,” and “the” include plural referents unless the content explicitly indicates otherwise. Thus, for example, reference to “an element” or “a element” includes a combination of two or more elements, notwithstanding use of other terms and phrases for one or more elements, such as “one or more.” The term “or” is, unless indicated otherwise, non-exclusive, i.e., encompassing both “and” and “or.” Terms describing conditional relationships, e.g., “in response to X, Y,” “upon X, Y,”, “if X, Y,” “when X, Y,” and the like, encompass causal relationships in which the antecedent is a necessary causal condition, the antecedent is a sufficient causal condition, or the antecedent is a contributory causal condition of the consequent, e.g., “state X occurs upon condition Y obtaining” is generic to “X occurs solely upon Y” and “X occurs upon Y and Z.” Such conditional relationships are not limited to consequences that instantly follow the antecedent obtaining, as some consequences may be delayed, and in conditional statements, antecedents are connected to their consequents, e.g., the antecedent is relevant to the likelihood of the consequent occurring. Statements in which a plurality of attributes or functions are mapped to a plurality of objects (e.g., one or more processors performing steps A, B, C, and D) encompasses both all such attributes or functions being mapped to all such objects and subsets of the attributes or functions being mapped to subsets of the attributes or functions (e.g., both all processors each performing steps A-D, and a case in which processor 1 performs step A, processor 2 performs step B and part of step C, and processor 3 performs part of step C and step D), unless otherwise indicated. Further, unless otherwise indicated, statements that one value or action is “based on” another condition or value encompass both instances in which the condition or value is the sole factor and instances in which the condition or value is one factor among a plurality of factors. Unless otherwise indicated, statements that “each” instance of some collection have some property should not be read to exclude cases where some otherwise identical or similar members of a larger collection do not have the property, i.e., each does not necessarily mean each and every. Limitations as to sequence of recited steps should not be read into the claims unless explicitly specified, e.g., with explicit language like “after performing X, performing Y,” in contrast to statements that might be improperly argued to imply sequence limitations, like “performing X on items, performing Y on the X'ed items,” used for purposes of making claims more readable rather than specifying sequence. Statements referring to “at least Z of A, B, and C,” and the like (e.g., “at least Z of A, B, or C”), refer to at least Z of the listed categories (A, B, and C) and do not require at least Z units in each category. Unless specifically stated otherwise, as apparent from the discussion, it is appreciated that throughout this specification discussions utilizing terms such as “processing,” “computing,” “calculating,” “determining” or the like refer to actions or processes of a specific apparatus, such as a special purpose computer or a similar special purpose electronic processing/computing device.

### APPENDIX

### Executive Summary: Audio Display of Lung Ultrasound for Layperson Recognition of Cardiopulmonary Disease

Introduction—The empowerment of the public to recognize disease through artificial intelligence (AI) methods has the potential to improve healthcare. Ultrasound devices can display early signs of life-threatening illness, such as the “B-lines” of lung congestion, that are not detectable by the stethoscope and chest X-ray. But as B-lines require skilled interpretation, AI efforts so far have focused solely on aiding the physician.

Our Solution—We have developed an AI method in which laypersons can identify B-lines using a simplified audio output. At Scripps Mercy Hospital, Dr. Bruce Kimura has pioneered a 25-year research effort in teaching ultrasound and founded one of the most established point-of-care ultrasound (POCUS) programs for internal medicine residency. With over 50 publications, Dr. Kimura's lab focuses on how POCUS is learned and utilizes an endless supply of patients and medical residents for validation studies. Last year, our lab published the feasibility of patient “self-imaging.” Now we have completed a proof-of-concept study for patient image interpretation using an audio AI model—11 untrained laypersons were able to detect B-lines by listening to sounds as accurately as 22 trained physicians were able to read them.

Opportunity and Value—Audio outputs that simplify ultrasound can enable both untrained healthcare providers and laypersons to improve healthcare costs and referral. For example, heart failure readmissions could be reduced by self-detection and early treatment of B-lines after discharge, saving a hospital $220,000/year in Medicare penalties alone. In COVID infection, B-line detection from home isolation can determine who needs expensive anti-viral therapies, potentially reducing prescription costs by $1 billion/year. With self-imaging, sound output from AI algorithms represents an entirely new method of rapid physical examination, home monitoring, community and outreach screening, or “wellness” assessment. The development of sound-emitting POCUS devices would unlock a large market, encompassing the $600 million stethoscope market and the $1.5 billion home BP monitor market, and offer more diagnostic capabilities than telemetry devices or Doppler patches. Initial use in healthcare will create familiarity for home application.

Conclusions and Next Steps—Our sense of hearing can alert, discriminate, and localize, making audio output of ultrasound data a novel blend of natural human skills with AI technology. Sound algorithms will need validation using existing POCUS equipment during concurrent hardware development of small audio devices envisioned for self-application. Our lab looks forward to furthering AI algorithms and clinical validation for a wide range of medical practice and home use in a collaborative effort to create a simple tool for a new paradigm in healthcare.

### An Artificial Intelligence Method Using Audio Display for Layperson Recognition of Pulmonary Edema or COVID Lung Infection on Ultrasound Images

Background: Although sound can be an effective alarm, few data exist on the use of audio output from an artificial intelligence program to simplify the detection of an abnormal lung ultrasound scan. We tested the accuracy of untrained laypersons to detect ultrasound B-line abnormalities when using a deep learning algorithm that produced sound.

Methods: A supervised deep learning convolutional neural network that assigned audio signals to represent the severity of B-lines was trained on 69 heart failure ultrasound studies. Diagnostic accuracy for one-or-more B-lines was tested on 11 laypersons solely listening to the model's output and compared to standard visual interpretation by 22 trained physicians, using video clips from 35 patients with suspected pulmonary edema and 23 outpatients with mild-moderate COVID infection.

Results: Of n=110 test video clips, B-lines were present in 43/110 (39%). Laypersons using the audio-based AI model successfully classified the presence of any B-lines with similar accuracy as the physician group: 79% vs. 80% accuracy (p=0.85), 84% vs. 94% sensitivity (p=0.07), 76% vs. 71% specificity (p=0.42), respectively. The presence or absence of an audio output was correctly identified in 100% of cases. The model showed no significant difference in accuracy, 81% vs. 77% (p=0.62) when applied to video clips from pulmonary edema vs COVID patients.

Conclusions: Lung ultrasound B-lines, whether formed in pulmonary edema or outpatient COVID infection, can be detected by laypersons using a deep learning model that produces an audible output. These findings may simplify patient self-examination from home and may foster novel ultrasound applications in medicine.

### Introduction

Our sense of hearing evolved to alarm and localize (1). In medicine, listening for abnormal sounds produced by the chest cavity has been a time-honored method to find and characterize lung pathology, since Hippocrates' succussion splash 2000 years ago and Laennec's mediate auscultation 200 years ago. Modern-day use of ultrasound technology can display pathologic lung B-lines, imaging artifacts that represent interstitial edema, relate to mortality, and often precede audible findings (2-4). Despite multiple studies demonstrating the prognostic value of B-lines in congestive heart failure (CHF)(4-9) and in COVID-19 infection (10-15), the examination for B-lines is still a relatively new practice in medicine with minimal data from ambulatory populations.

Artificial intelligence (AI) methods applied to data from small, wearable devices can alert the outpatient of some cardiac disorders, such as paroxysmal atrial fibrillation, providing the capability for home screening and remote monitoring. As B-line recognition requires skilled and semi-quantitative visual interpretation, current AI development in lung ultrasound has focused on aiding physicians in the detection, counting, and mapping of multiple B-lines on video images acquired from acutely ill patients. Few data exist on the use of an audio output from AI methods to simplify the early detection of an abnormal scan for application by the patients themselves. To capitalize on hearing as a natural alert system, we tested the accuracy of a deep learning algorithm that produced sound to represent B-lines when used by untrained laypersons.

### Methods

An AI classifier using a supervised deep learning convolutional neural network was trained on 7772 and validated on 2072 lung images using 69 de-identified videos obtained on patients hospitalized with suspected CHF. The study was approved by the Scripps Institutional Review Board (Scripps Health, San Diego). All images had been obtained with pocket-sized ultrasound devices (Lumify, Phillips Healthcare, Bothell, WA) using 3 MHz cardiac transducers and preset, acquired in the longitudinal plane in the second intercostal space in the mid-clavicular line as in previous imaging protocols (2,15-18), and stored as 3-second video files in mp4 format (1024×768 resolution) by the Lumify device. Each of the video clips had been evaluated by an expert physician with over 15 years of clinical and research experience in lung ultrasound for B lines, artifacts defined as vertical lung artifacts that arise from the pleural line and extend toward the bottom of the screen (2,3). Primarily based on the number of B-lines, the video clips represented a spectrum of severity, providing the model with training on balanced proportions of normal, mild, moderate, and severe disease. The expert physician categorized disease severity on each frame within each video clip as normal with no B-lines, mild-moderately abnormal with 1-2 B-lines, and severely abnormal, having 3-or-more or coalesced B-lines. Frames were masked to display only the imaging sector for training the model. Within the 3-second clip, the model evaluated sequential batches of frames and assigned chirping sounds in relation to the model's classification of disease severity. Qualitatively, the model's audio playback was designed to sound like the audio output of a Geiger counter, emitting multiple chirps, up to 9 per second, as the number of B-lines increased in the video.

The AI model was then tested when used by laypersons and compared to physician interpretation on video images from CHF and COVID-19 referrals, two groups in which evidence supports the value of B-line detection. A separate clinical lung ultrasound video dataset of 110 technically adequate de-identified studies was compiled consisting of 67 video clips from 35 hospitalized patients with suspected decompensated CHF from inpatient databases and 43 video clips from 23 consecutive outpatients with mild-moderate COVID-19 infection from an outpatient monoclonal antibody infusion clinic database. Each of the video clips, representing unilateral lung apical images had been evaluated by the same expert for the presence of B-lines which served as the reference standard. Video clips were evaluated by the model, transformed into a 3-second audio file, and then randomized in their order to avoid bias. A group of 11 untrained layperson volunteers (5 were nonmedical, 6 were medical office workers) were each asked to listen to a different 10-audio clip sample and report whether they heard any sound when the clip was played through the speaker of a laptop computer. No layperson had previous ultrasound training or experience and no video images were displayed. All 110 audio clips were evaluated by this layperson-audio group (11 persons listened to 10 audios each). The collective accuracy of this group was compared to the collective accuracy of the visual interpretations of the 110-video clip database video clips by a group of 22 physicians, each reading a sample of 10 video clips, resulting in the entire database being read twice (22 physicians read 10 video clips each). The 10-case allotment was intended to reduce reading fatigue and provide a larger sample of physician readers. Physicians were asked to report if they saw 1-or-more B-lines in the continuously playing video loops. This physician-visual group was comprised of senior internal medicine residents (n=19) or attendings (n=3) who had graduated from a year-long course, passed a proficiency exam, and continued to use lung imaging routinely in their practice afterward, having a median of 2 years (range: 1-14 years) of clinical lung imaging. All physicians had been previously taught and tested for proficiency in lung imaging in an established training program (19) by the same expert physician whose interpretations were used to train the model.

Diagnostic accuracy was computed for the layperson-audio group and the physician-visual group for one-or-more B-lines, using the interpretation of the expert physician as the reference standard. The expert's intra-observer variability was assessed by having the expert re-interpret the entire database of 110 video clips after a four-week interval had passed. Algorithm errors were categorized as false positive or false negative and were attributed to (1) misclassification by the model, (2) technically difficult or inadequate images, or (3) equivocal or “borderline” findings, using frame-by-frame review by the expert physician and computer programmer.

Data are represented as mean±standard deviation. Sensitivity, specificity, and total accuracy were expressed as percentages and computed with 95% confidence intervals [CI] computed for each parameter. Intra-observer agreement was assessed using Cohen's kappa with computed CI. Because of multiple images and assessments per patient, non-parametric bootstrap with per-patient resampling was used to compute all CIs and to compare the performance parameters between the layperson and volunteer group and between pulmonary edema and COVID-19 data. Statistical analyses were performed using R: R Core Team (2018). R: A language and environment for statistical computing. R Foundation for Statistical Computing, Vienna, Austria. URL https://www.R-project.org/. A p<0.05 was considered significant.

### Results

Of n=110 video clips used in the comparison, 67 (61%) were categorized as normal, 36 (33%) were mild-moderate disease, and 7 (6%) were severe, showing 3-or-more B-lines or their confluence. B-lines were present in 33/67 (49%) of the inpatient CHF group and 10/43 (23%) in the outpatient group with mild-moderate COVID-19. Laypersons using the audio-based AI model successfully classified the presence of one-or-more B-lines similar to the physician-visual group: accuracy of 79% [95% CI: 69%-86%] vs. 80% [95% CI: 72%-86%] (p=0.85), sensitivity of 84% [95% CI: 69%-93%] vs. 94% [95% CI: 85%-99%] (p=0.07), and specificity of 76% [95% CI: 63%-86%] vs. 71% [95% CI: 61%-80%] (p=0.42), respectively. The model's accuracy was not significantly different, 81% [95% CI: 67%-90%] vs. 77% [95% CI: 62%-88%] (p=0.62) when applied to images from pulmonary edema vs. from COVID-19 patients. The physician-visual accuracy was higher for pulmonary edema than the physician-visual accuracy for COVID-19, 86% [95% CI: 76%-92%] vs. 71% [95% CI: 57%-82%] (p=0.02) (see Figure). No misinterpretation of the audio output occurred in laypersons, and therefore the layperson accuracy solely reflected the accuracy of the AI classifier at detecting disease. In other words, laypersons detected 100% of audio clips that produced a sound, even when the 3-second clip contained only 2-or-less chirps, noted in 9 cases, and demonstrated no cases of falsely hearing a sound.

The physician-visual group showed high sensitivity, with only 6 false negatives, but low specificity, with 40 false positives, in detecting 1 or more B-lines. Of 22 individual physicians, interpretative errors occurred at a mean of 2.2±2.0 errors/10 videos. The expert interpretation showed an intra-observer agreement of 95%, a Cohen's kappa of 0.89 [95% CI: 0.78-0.96], with 6/110 discrepant interpretations, later found on re-review to occur primarily on equivocal cases.

In the AI error analysis, of n=23 errors by the model (16 false positives, 7 false negatives), 79% were misclassified, 4% were technically difficult, and 17% were considered equivocal findings or borderline cases. The majority of misclassified cases were false positives (13/19), of which 10 were due to regions of oversaturation interpreted as a confluence of B-lines.

### Discussion

This proof-of-concept investigation found that laypersons using an AI model with an audio output showed no significant difference in B-line detection compared to trained physicians interpreting video images. Since B-lines are early findings with an adverse prognosis in patients with suspected pulmonary edema or mild-moderate COVID-19 infection, our findings from these specific groups suggest that audio AI methods may enable patient self-examination from home after hospital discharge or during isolation, to risk stratify, monitor, or alter therapies. As AI approaches become more widely accepted, sound output may be a novel method to simplify and portray complex ultrasound abnormalities for recognition by untrained individuals.

The B-line, a “ring-down” reverberation artifact that emanates from the visceral pleural line on lung ultrasound and proceeds vertically to the bottom of the screen, is felt to be due to entrapment of the ultrasound beam in moist or collagenous superficial interstitial spaces (2-4). In the development of pulmonary edema, interstitial edema precedes the clinical manifestations of alveolar flooding. Accordingly, B-lines form before rales on exam, hypoxemia on pulse oximetry, or infiltrates on chest radiographs (20). Multiple studies have shown a relationship of B-lines with worse outcomes whether found in hospitalized patients admitted with CHF (5-9), COVID-19 infection (10-15), or in those simply referred for echocardiography (18) or hospitalized for cardiac disease (21).

Involving patients in their own health can potentially reduce medical costs while improving care. In CHF, approximately 20% of hospital discharges are readmitted within 3 months, an observation that has resulted in penalties from the Hospital Readmissions Reduction Program to contain Medicare expenditures (22). The recognition of B-line recurrence in the weeks after discharge from home could herald relapse of pulmonary edema even before symptoms or hypoxemia can alert the healthcare provider, allowing more time to treat and avert readmission. In COVID-19, the presence of B-lines is related to the severity of disease and mortality. In a study of mild-moderate outpatient COVID-19infection (17), a significant percentage, 27%, of the 201 patients receiving monoclonal antibody infusion demonstrated lung apical B-lines in the absence of oxygen desaturation and were able to display the finding over telehealth. As patients during this time are in home isolation, “self-detection” of B-lines can signal disease progression and justify the costs of the antiviral therapies that have been shown to reduce mortality and hospitalization. In both CHF and COVID-19, the capability for a patient to self-image by placing the probe in the antero-apical chest has been demonstrated, either by written instruction (16) or by telehealth guidance (17). Although modest and easily performed on one's self or family member within seconds, the exam still requires a trained physician to interpret its complex findings. However, given the comparable diagnostic accuracy of the layperson-audio and physician-video groups in the current study, outpatient surveillance for B-lines could now potentially occur using AI-equipped, audio output devices kept with the patient. Similar to event monitoring, such devices could be applied daily while performing exercise or at the time of dyspnea to listen for inducible interstitial edema or during upper respiratory infections to listen for progression to pneumonitis.

The use of an audio display from an artificial intelligence model is a new paradigm in the detection of pulmonary edema, adding sound to early findings that are inaudible by auscultation. Previous work in automated B-line image recognition has resulted in machine learning models that can be taught to recognize and count B-line numbers or enhance their display (23-26). But as hearing is an innate, discriminating, alarm-localization system, sound output may have several natural advantages compared to visual representation, especially for the untrained layperson (1). Our first-generation model reaffirms at the most basic level the natural human ability to be alerted by sound, as none of the 11 untrained volunteers misheard the model's outputs, which was the most rudimentary, single-intensity chirp that was emitted when a B-line was recognized by the model. In the future, modulation and assignment of sound intensity, duration, tempo, timbre, pitch, and sequencing could be programmed to represent the variability in brightness, penetration, patterns of coalescence, and respiratory variations of B-lines. Sound assignments could also localize pathology, as exemplified by the cluttering of sounds of a Geiger counter or squeal of a metal detector, with sites being remembered more readily than when displayed by numerical or visual outputs (27,28). The ease with which a sound algorithm can be programmed and uploaded underscores the potential of a small device to emit tones updated to guide transducer location to a specific region for diagnosis and has implications for the burgeoning field of AI-guided ultrasound data acquisition, tissue characterization, and interpretation.

As a proof-of-concept, our study has limitations. Model learning could potentially improve by using the radiofrequency signal prior to the pre-and post-processing and data compression employed to create the two-dimensional image. The study used data from one manufacturer's device but has no reason to believe that the model, if trained similarly, would fail its primary goal of sound assignment using other platforms or even other AI algorithms. To focus on the model's sensitivity, the analysis used the presence of only one B-line as the minimal abnormal criterion for producing a sound. We realize that using the audio equivalent of the conventional “visual” criteria of 3 B-lines being simultaneously present in a single view would have improved specificity and could be simply achieved by a different sonification protocol. To our knowledge, our AI model analysis employs perhaps the largest population in the literature in which both the physicians and the model were trained by the same expert and their accuracies compared, providing unique insight into human and machine learning processes, such as the biases that increase human sensitivity. Nonetheless, the current study was likely underpowered to maximize model accuracy, determine equivalency, or detect differences between lung image patterns from COVID-19 and heart failure as previously shown (29) and the one significant finding, without adjustment for multiple comparisons, may have been spurious. This initial version of the audio model did demonstrate diagnostic performance on validation and testing datasets comparable to previous studies (25), and we anticipate that with more training, the model could improve layperson accuracy to surpass that of the physician group. Future studies may wish to modulate the sonification algorithm and compare to the current, conventional visual criteria when predicting disease severity and, ultimately, patient outcome.

To our knowledge, our study is the first to investigate sound output from an artificial intelligence algorithm and its application to layperson ultrasound use. The assignment of sound to ultrasound data via artificial intelligence methods may represent a new field within translational medicine that could combine technology with the natural advantages of human hearing.

Diagnostic parameters of sensitivity, specificity, accuracy and values have been shown by group, laypersons-audio and physicians-visual, from patients with COVID-19 and suspected CHF have shown similar diagnostic accuracy. Note that physician visual accuracy of CHF was significantly different from COVID (86% vs. 71%) (p<0.05).

### REFERENCES

1. Hermann, T., Hunt, A., Neuhoff, J. G. (2011). Introduction. In Hermann, T., Hunt, A., Neuhoff, J. G., editors, The Sonification Handbook, chapter 1, pages 1-6. Logos Publishing House, Berlin, Germany.
2. Volpicelli G, Elbarbary M, Blaivas M, et al. International Liaison Committee on Lung Ultrasound (ILC-LUS) for International Consensus Conference on Lung Ultrasound (ICC-LUS). International evidence-based recommendations for point-of-care lung ultrasound. Intensive Care Med. 2012 April; 38(4):577-91.
3. Demi L, Wolfram F, Klersy C, et al. New International Guidelines and Consensus on the Use of Lung Ultrasound. J Ultrasound Med. 2023 February; 42(2):309-344.
4. Picano E, Frassi F, Agricola E, Gligorova S, Gargani L, Mottola G. Ultrasound lung comets: a clinically useful sign of extravascular lung water. J Am Soc Echocardiogr. 2006 March; 19(3):356-63.
5. Goonewardena S N, Gemignani A, Ronan A, et al. Comparison of hand-carried ultrasound assessment of the inferior vena cava and N-terminal pro-brain natriuretic peptide for predicting readmission after hospitalization for acute decompensated heart failure. JACC Cardiovasc Imaging. 2008 September; 1(5):595-601.
6. Cogliati C, Casazza G, Ceriani E, et al. Lung ultrasound and short-term prognosis in heart failure patients. Int J Cardiol. 2016 Sep. 1; 218:104-108.
7. Miglioranza M H, Picano E, Badano L P, et al. Pulmonary congestion evaluated by lung ultrasound predicts decompensation in heart failure outpatients. Int J Cardiol. 2017 Aug. 1; 240:271-278.
8. Platz E, Merz A A, Jhund P S, Vazir A, Campbell R, McMurray J J. Dynamic changes and prognostic value of pulmonary congestion by lung ultrasound in acute and chronic heart failure: a systematic review. Eur J Heart Fail. 2017 Sep; 19(9):1154-1163.
9. Coiro S, Porot G, Rossignol P, et al. Prognostic value of pulmonary congestion assessed by lung ultrasound imaging during heart failure hospitalisation: A two-centre cohort study. Sci Rep. 2016 Dec. 20; 6:39426.
10. Casella F, Barchiesi M, Leidi F, et al. Lung ultrasonography: A prognostic tool in non-ICU hospitalized patients with COVID-19 pneumonia. Eur J Intern Med. 2021 March; 85:34-40.
11. Sun Z, Zhang Z, Liu J, et al. Lung Ultrasound Score as a Predictor of Mortality in Patients With COVID-19. Front Cardiovasc Med. 2021 May 25; 8:633539.
12. de Alencar J C G, Marchini J F M, Marino L O, et al. Lung ultrasound score predicts outcomes in COVID-19 patients admitted to the emergency department. Ann Intensive Care. 2021 Jan. 11; 11(1):6.
13. Mafort T T, Rufino R, da Costa C H, et al. One-month outcomes of patients with SARS-CoV-2 infection and their relationships with lung ultrasound signs. Ultrasound J. 2021 Apr. 9; 13(1): 19.
14. Lichter Y, Topilsky Y, Taieb P, et al. Lung ultrasound predicts clinical course and outcomes in COVID-19 patients. Intensive Care Med. 2020 October; 46(10):1873-1883.
15. Kimura B J, Shi R, Tran E M, Spierling Bagsic S R, Resnikoff P M. Outcomes of Simplified Lung Ultrasound Exam in COVID-19: Implications for Self-Imaging. J Ultrasound Med. 2022 June; 41(6):1377-1384.
16. Resnikoff P M, Shi R, Spierling Bagsic S R, Kimura B J. The Novel Concept of Patient Self-Imaging: Success in COVID-19 and Cardiopulmonary Disorders. Am J Med. 2021 May; 134(5):e360-e361.
17. Kimura B J, Resnikoff P M, Tran E M, Bonagiri P R, Spierling Bagsic S R. Simplified Lung Ultrasound Examination and Telehealth Feasibility in Early SARS-COV-2 Infection. J Am Soc Echocardiogr. 2022 October; 35(10):1047-1054.
18. Garibyan V N, Amundson S A, Shaw D J, Phan J N, Showalter B K, Kimura B J. Lung Ultrasound Findings Detected During Inpatient Echocardiography Are Common and Associated with Short- and Long-term Mortality. J Ultrasound Med. 2018 July; 37(7):1641-1648.
19. Kimura B J, Amundson S A, Phan J N, Agan D L, Shaw D J. Observations during development of an internal medicine residency training program in cardiovascular limited ultrasound examination. J Hosp Med. 2012 September; 7(7):537-42.
20. Torino C, Gargani L, Sicari R, et al. The Agreement between Auscultation and Lung Ultrasound in Hemodialysis Patients: The LUST Study. Clin J Am Soc Nephrol. 2016 Nov. 7; 11(11):2005-2011.
21. Gargani L, Pugliese N R, Frassi F, et al. Prognostic value of lung ultrasound in patients hospitalized for heart disease irrespective of symptoms and ejection fraction. ESC Heart Fail. 2021 August; 8(4):2660-2669.
22. Zuckerman R B, Sheingold S H, Orav E J, Ruhter J, Epstein A M. Readmissions, Observation, and the Hospital Readmissions Reduction Program. N Engl J Med. 2016 Apr. 21; 374(16):1543-51.
23. Moore C L, Wang J, Battisti A J, et al. Interobserver Agreement and Correlation of an Automated Algorithm for B-Line Identification and Quantification With Expert Sonologist Review in a Handheld Ultrasound Device. J Ultrasound Med. 2022 October; 41(10):2487-2495.
24. Russell F M, Ehrman R R, Barton A, Sarmiento E, Ottenhoff J E, Nti B K. B-line quantification: comparing learners novice to lung ultrasound assisted by machine artificial intelligence technology to expert review. Ultrasound J. 2021 Jun. 30; 13(1):33.
25. Mento F, Khan U, Faita F, et al. State of the Art in Lung Ultrasound, Shifting from Qualitative to Quantitative Analyses. Ultrasound Med Biol. 2022 December; 48(12):2398-2416.
26. Arntfield R, Wu D, Tschirhart J, et al. Automation of Lung Ultrasound Interpretation via Deep Learning for the Classification of Normal versus Abnormal Lung Parenchyma: A Multicenter Study. Diagnostics (Basel). 2021 Nov. 4; 11(11):2049.
27. Inui K, Urakawa T, Yamashiro K, et al. Echoic memory of a single pure tone indexed by change-related brain activity. BMC Neurosci. 2010 Oct. 20; 11:135.
28. Nees M A. Have We Forgotten Auditory Sensory Memory? Retention Intervals in Studies of Nonverbal Auditory Working Memory. Front Psychol. 2016 Dec. 2; 7:1892.
29. Arntfield R, VanBerlo B, Alaifan T, et al. Development of a convolutional neural network to differentiate among the etiology of similar appearing pathological B lines on lung ultrasound: a deep learning study. BMJ Open. 2021 Mar. 5; 11(3):e045120.

What is claimed is:

1. A system configured to provide sensory output indicating lung thickening in a subject, the system comprising:
a sensor configured to move proximate to one or both lungs of the subject, the sensor configured to generate one or more output signals comprising information indicating areas of lung thickening due to congestion, infection, inflammation, and/or fibrosis;
a sensory output device configured to generate sensory output indicating the areas of lung thickening as the sensor moves proximate to the lungs; and
one or more processors configured by machine readable instructions to:
execute a trained machine learning model to detect the areas of lung thickening based on the output signals; and
control the sensory output device to generate and/or modulate the sensory output at spatially localized areas of lung thickening when the sensor moves proximate to the areas of lung thickening.

2. The system of claim 1, wherein the sensor comprises an ultrasound apparatus.

3. The system of claim 1, where detecting the areas of lung thickening based on the output signals, and controlling the sensory output device to generate and/or modulate the sensory output at the areas of lung thickening, occurs in real time as the sensor moves proximate to the areas of lung thickening in the subject.

4. The system of claim 1, wherein sensor movement proximate to one or both lungs of the subject comprises a back and forth rastering motion across the lungs.

5. The system of claim 1, wherein the sensory output comprises:
auditory output including one or more sounds generated by the sensory output device;
haptic output including one or more vibrations or patterns of vibrations generated by the sensory output device; and/or
visual output including one or more flashes or patterns of flashes of light, and/or one or more colors or patterns of colors of light, generated by the sensory output device.

6. The system of claim 1, wherein the sensory output intensifies or otherwise modulates as the sensor moves closer to an area of lung thickening.

7. The system of claim 1, wherein the sensory output comprises auditory output, and the auditory output comprises a series of clicks configured to intensify as the sensor moves closer to an area of lung thickening.

8. The system of claim 7, wherein intensification comprises an increase in click volume and/or click frequency.

9. The system of claim 1, wherein the one or more output signals comprise, and/or are used to generate,
raw radiofrequency data,
one or more images of the lungs of the subject,
pre and post processed scan lines, and/or
a constructed postprocessed 2D image.

10. The system of claim 1, wherein the one or more output signals indicate B-lines associated with the lungs of the subject, and wherein the trained machine learning model is trained to detect the areas of lung thickening based on the B-lines.

11. The system of claim 1, wherein the trained machine learning model is trained by:
obtaining and providing prior sensor output signals associated with areas of lung thickening to the machine learning model; wherein portions of the prior sensor output signals associated with the areas of lung thickening are labeled as thickened lung areas.

12. The system of claim 1, wherein the trained machine learning model is trained with training data, the training data comprising input output training pairs comprising a labeled B-line in an output signal and a corresponding indication of spatially localized area of the lungs.

13. The system of claim 1, wherein the sensory output is configured to facilitate detection and localization of abnormalities associated with lung thickening in the lungs.

14. The system of claim 1, wherein the trained machine learning model is trained based on database ultrasound images that have been ranked according to B-line number and echogenicity, representing diagnostic certainty for an abnormal image such that the more numerous and distinct the B-lines appear on an image the more recognizable the sensory output.

15. The system of claim 14, wherein the database ultrasound images comprise extracted individual frames from a video, where an annotation tool has been applied to visualize and/or label relevant properties of a frame, and where, for image classification, relevant properties include an estimated severity score, an annotator's confidence score for the estimated severity score, a number of B-lines, and/or a number of A-lines.

16. The system of claim 1, wherein the trained machine learning model is configured to use temporal characteristics of training data including disappearance of B-lines during respiration and data averaging to assess severity of lung thickening, so as to avoid errors due to evanescence or confluence of B-lines.

17. The system of claim 1, wherein the trained machine learning model is trained on a dataset that is uniquely interpreted cognizant to a finding's relationship to diagnostic certainty, such that a diagnostic accuracy and clinical utility of the system is higher than prior systems.

18. The system of claim 1, wherein the sensor, the sensory output device, and the one or more processors comprise a wearable device, and wherein the wearable device is configured to be worn at or near an area of lung thickening.

19. The system of claim 1, wherein the machine learning model comprises a deep neural network.

20. The system of claim 1, wherein the sensor is configured to be moved by the subject.

21. A method for providing sensory output indicating lung thickening in a subject, the method comprising:
moving a sensor proximate to one or both lungs of the subject, the sensor configured to generate one or more output signals comprising information indicating areas of lung thickening due to congestion, infection, inflammation, and/or fibrosis;
generating, with a sensory output device, sensory output indicating the areas of lung thickening as the sensor moves proximate to the lungs;
executing, with one or more processors configured by machine readable instructions, a trained machine learning model to detect the areas of lung thickening based on the output signals; and
controlling, with the one or more processors, the sensory output device to generate and/or modulate the sensory output at spatially localized areas of lung thickening when the sensor moves proximate to the areas of lung thickening.

22. The method of claim 21, wherein the sensor comprises an ultrasound apparatus.

23. The method of claim 21, wherein detecting the areas of lung thickening based on the output signals, and controlling the sensory output device to generate and/or modulate the sensory output at the areas of lung thickening, occurs in real time as the sensor moves proximate to the areas of lung thickening in the subject.

24. The method of claim 21, wherein sensor movement proximate to one or both lungs of the subject comprises a back and forth rastering motion across the lungs.

25. The method of claim 21, wherein the sensory output comprises:
auditory output including one or more sounds generated by the sensory output device;
haptic output including one or more vibrations or patterns of vibrations generated by the sensory output device; and/or
visual output including one or more flashes or patterns of flashes of light, and/or one or more colors or patterns of colors of light, generated by the sensory output device.

26. The method of claim 21, wherein the sensory output intensifies or otherwise modulates as the sensor moves closer to an area of lung thickening.

27. The method of claim 21, wherein the sensory output comprises auditory output, and the auditory output comprises a series of clicks configured to intensify as the sensor moves closer to an area of lung thickening.

28. The method of claim 27, wherein intensification comprises an increase in click volume and/or click frequency.

29. The method of claim 21, wherein the one or more output signals comprise, and/or are used to generate,
raw radiofrequency data,
one or more images of the lungs of the subject,
pre and post processed scan lines, and/or
a constructed postprocessed 2D image.

30. The method of claim 21, wherein the one or more output signals indicate B-lines associated with the lungs of the subject, and wherein the trained machine learning model is trained to detect the areas of lung thickening based on the B-lines.

31. The method of claim 21, wherein the trained machine learning model is trained by:
obtaining and providing prior sensor output signals associated with areas of lung thickening to the machine learning model; wherein portions of the prior sensor output signals associated with the areas of lung thickening are labeled as thickened lung areas.

32. The method of claim 21, wherein the trained machine learning model is trained with training data, the training data comprising input output training pairs comprising a labeled B-line in an output signal and a corresponding indication of spatially localized area of the lungs.

33. The method of claim 21, wherein the sensory output is configured to facilitate detection and localization of abnormalities associated with lung thickening in the lungs.

34. The method of claim 21, wherein the trained machine learning model is trained based on database ultrasound images that have been ranked according to B-line number and echogenicity, representing diagnostic certainty for an abnormal image such that the more numerous and distinct the B-lines appear on an image the more recognizable the sensory output.

35. The method of claim 34, wherein the database ultrasound images comprise extracted individual frames from a video, where an annotation tool has been applied to visualize and/or label relevant properties of a frame, and where, for image classification, relevant properties include an estimated severity score, an annotator's confidence score for the estimated severity score, a number of B-lines, and/or a number of A-lines.

36. The method of claim 21, wherein the trained machine learning model is configured to use temporal characteristics of training data including disappearance of B-lines during respiration and data averaging to assess severity of lung thickening, so as to avoid errors due to evanescence or confluence of B-lines.

37. The method of claim 21, wherein the trained machine learning model is trained on a dataset that is uniquely interpreted cognizant to a finding's relationship to diagnostic certainty, such that a diagnostic accuracy and clinical utility of the method is higher than prior systems.

38. The method of claim 21, wherein the sensor, the sensory output device, and the one or more processors comprise a wearable device, and wherein the wearable device is configured to be worn at or near an area of lung thickening.

39. The method of claim 21, wherein the machine learning model comprises a deep neural network.

40. The method of claim 21, wherein the sensor is configured to be moved by the subject.

41. A non-transitory computer readable medium having instructions thereon, the instructions when executed by a computer causing the computer to perform operations comprising:
receiving, from a sensor that is moved proximate to one or both lungs of a subject, one or more output signals comprising information indicating areas of lung thickening due to congestion, infection, inflammation, and/or fibrosis;
executing a trained machine learning model to detect the areas of lung thickening based on the output signals; and
controlling a sensory output device to generate and/or modulate sensory output at spatially localized areas of lung thickening when the sensor moves proximate to the areas of lung thickening.

42. The medium of claim 41, wherein the sensor comprises an ultrasound apparatus.

43. The medium of claim 41, where detecting the areas of lung thickening based on the output signals, and controlling the sensory output device to generate and/or modulate the sensory output at the areas of lung thickening, occurs in real time as the sensor moves proximate to the areas of lung thickening in the subject.

44. The medium of claim 41, wherein sensor movement proximate to one or both lungs of the subject comprises a back and forth rastering motion across the lungs.

45. The medium of claim 41, wherein the sensory output comprises:
auditory output including one or more sounds generated by the sensory output device;
haptic output including one or more vibrations or patterns of vibrations generated by the sensory output device; and/or
visual output including one or more flashes or patterns of flashes of light, and/or one or more colors or patterns of colors of light, generated by the sensory output device.

46. The medium of claim 41, wherein the sensory output intensifies or otherwise modulates as the sensor moves closer to an area of lung thickening.

47. The medium of claim 41, wherein the sensory output comprises auditory output, and the auditory output comprises a series of clicks configured to intensify as the sensor moves closer to an area of lung thickening.

48. The medium of claim 47, wherein intensification comprises an increase in click volume and/or click frequency.

49. The medium of claim 41, wherein the one or more output signals comprise, and/or are used to generate,
raw radiofrequency data,
one or more images of the lungs of the subject,
pre and post processed scan lines, and/or
a constructed postprocessed 2D image.

50. The medium of claim 41, wherein the one or more output signals indicate B-lines associated with the lungs of the subject, and wherein the trained machine learning model is trained to detect the areas of lung thickening based on the B-lines.

51. The medium of claim 41, wherein the trained machine learning model is trained by:
obtaining and providing prior sensor output signals associated with areas of lung thickening to the machine learning model; wherein portions of the prior sensor output signals associated with the areas of lung thickening are labeled as thickened lung areas.

52. The medium of claim 41, wherein the trained machine learning model is trained with training data, the training data comprising input output training pairs comprising a labeled B-line in an output signal and a corresponding indication of spatially localized area of the lungs.

53. The medium of claim 41, wherein the sensory output is configured to facilitate detection and localization of abnormalities associated with lung thickening in the lungs.

54. The medium of claim 41, wherein the trained machine learning model is trained based on database ultrasound images that have been ranked according to B-line number and echogenicity, representing diagnostic certainty for an abnormal image such that the more numerous and distinct the B-lines appear on an image the more recognizable the sensory output.

55. The medium of claim 54, wherein the database ultrasound images comprise extracted individual frames from a video, where an annotation tool has been applied to visualize and/or label relevant properties of a frame, and where, for image classification, relevant properties include an estimated severity score, an annotator's confidence score for the estimated severity score, a number of B-lines, and/or a number of A-lines.

56. The medium of claim 41, wherein the trained machine learning model is configured to use temporal characteristics of training data including disappearance of B-lines during respiration and data averaging to assess severity of lung thickening, so as to avoid errors due to evanescence or confluence of B-lines.

57. The medium of claim 41, wherein the trained machine learning model is trained on a dataset that is uniquely interpreted cognizant to a finding's relationship to diagnostic certainty, such that a diagnostic accuracy and clinical utility is higher than that of prior systems.

58. The medium of claim 41, wherein the sensor, the sensory output device, and the one or more processors comprise a wearable device, and wherein the wearable device is configured to be worn at or near an area of lung thickening.

59. The medium of claim 41, wherein the machine learning model comprises a deep neural network.

60. The medium of claim 41, wherein the sensor is configured to be moved by the subject.

61. A system configured to provide sensory output indicating an abnormality of interest in a target area in an organ of a subject, the system comprising:
a sensor configured to move proximate to the target area in the organ of the subject, the sensor configured to generate one or more output signals comprising information indicating (1) a location of the sensor relative to the target area, and (2) presence of the abnormality of interest at or near the target area;
a sensory output device configured to generate sensory output indicating (1) the location of the sensor relative to the target area, and (2) the presence of the abnormality of interest at or near the target area as the sensor moves proximate to the organ; and
one or more processors configured by machine readable instructions to:
execute a trained machine learning model to (1) determine the location of the sensor relative to the target area, and (2) detect the presence of the abnormality of interest, based on the output signals; and
control the sensory output device to generate and/or modulate the sensory output at or near the organ and/or the target area when the sensor moves proximate to the organ and/or the target area, the generating and/or modulating comprising:
causing the sensory output to modulate as the sensor is moved nearer to, or further from, the organ and/or the target area, such that the sensory output indicates a location of the target area to the subject; and
responsive to the sensor being located proximate to the organ and/or the target area, causing the sensory output to indicate spatially localized areas of the target area with the abnormality of interest when the sensor moves around the target area.

62. The system of claim 61, wherein the one or more processors are configured to execute machine readable instructions for determining the location of the sensor relative to the target area, detecting the presence of the abnormality of interest, and controlling the sensory output device to generate and/or modulate the sensory output, the machine readable instructions configured to be changeable based on the organ, the target area, and/or the abnormality of interest.

63. The system of claim 62, wherein the organ comprises a lung, a heart, or a liver of the subject.

64. The system of claim 61, wherein the sensor comprises an ultrasound apparatus, and wherein the sensory output comprises: auditory output including one or more sounds generated by the sensory output device; haptic output including one or more vibrations or patterns of vibrations generated by the sensory output device; and/or visual output including one or more flashes or patterns of flashes of light, and/or one or more colors or patterns of colors of light, generated by the sensory output device.

65. The system of claim 61, wherein the sensory output intensifies or otherwise modulates as the sensor moves closer to the abnormality of interest.

66. A method for providing sensory output indicating an abnormality of interest in a target area in an organ of a subject, the method comprising:
generating, with a sensor configured to move proximate to the target area in the organ of the subject, one or more output signals comprising information indicating (1) a location of the sensor relative to the target area, and (2) presence of the abnormality of interest at or near the target area;
generating, with a sensory output device, sensory output indicating (1) the location of the sensor relative to the target area, and (2) the presence of the abnormality of interest at or near the target area as the sensor moves proximate to the organ;
executing, with one or more processors, a trained machine learning model to (1) determine the location of the sensor relative to the target area, and (2) detect the presence of the abnormality of interest, based on the output signals; and
controlling, with the one or more processors, the sensory output device to generate and/or modulate the sensory output at or near the organ and/or the target area when the sensor moves proximate to the organ and/or the target area, the generating and/or modulating comprising:
causing the sensory output to modulate as the sensor is moved nearer to, or further from, the organ and/or the target area, such that the sensory output indicates a location of the target area to the subject; and
responsive to the sensor being located proximate to the organ and/or the target area, causing the sensory output to indicate spatially localized areas of the target area with the abnormality of interest when the sensor moves around the target area.

67. The method of claim 66, wherein the one or more processors are configured to execute machine readable instructions for determining the location of the sensor relative to the target area, detecting the presence of the abnormality of interest, and controlling the sensory output device to generate and/or modulate the sensory output, the machine readable instructions configured to be changeable based on the organ, the target area, and/or the abnormality of interest.

68. The method of claim 67, wherein the organ comprises a lung, a heart, or a liver of the subject.

69. The method of claim 66, wherein the sensor comprises an ultrasound apparatus, and wherein the sensory output comprises: auditory output including one or more sounds generated by the sensory output device; haptic output including one or more vibrations or patterns of vibrations generated by the sensory output device; and/or visual output including one or more flashes or patterns of flashes of light, and/or one or more colors or patterns of colors of light, generated by the sensory output device.

70. The method of claim 66, wherein the sensory output intensifies or otherwise modulates as the sensor moves closer to the abnormality of interest.

71. A non-transitory computer readable medium having instructions thereon, the instructions when executed by a computer causing the computer to perform operations comprising:
receiving, from a sensor configured to move proximate to a target area in an organ of a subject, one or more output signals comprising information indicating (1) a location of the sensor relative to the target area, and (2) presence of an abnormality of interest at or near the target area;
causing generation, with a sensory output device, of sensory output indicating (1) the location of the sensor relative to the target area, and (2) the presence of the abnormality of interest at or near the target area as the sensor moves proximate to the organ;
executing, with one or more processors, a trained machine learning model to (1) determine the location of the sensor relative to the target area, and (2) detect the presence of the abnormality of interest, based on the output signals; and
controlling, with the one or more processors, the sensory output device to generate and/or modulate the sensory output at or near the organ and/or the target area when the sensor moves proximate to the organ and/or the target area, the generating and/or modulating comprising:
causing the sensory output to modulate as the sensor is moved nearer to, or further from, the organ and/or the target area, such that the sensory output indicates a location of the target area to the subject; and
responsive to the sensor being located proximate to the organ and/or the target area, causing the sensory output to indicate spatially localized areas of the target area with the abnormality of interest when the sensor moves around the target area.

72. The medium of claim 71, wherein the instructions configured to be changeable based on the organ, the target area, and/or the abnormality of interest.

73. The medium of claim 72, wherein the organ comprises a lung, a heart, or a liver of the subject.

74. The medium of claim 71, wherein the sensor comprises an ultrasound apparatus, and wherein the sensory output comprises: auditory output including one or more sounds generated by the sensory output device; haptic output including one or more vibrations or patterns of vibrations generated by the sensory output device; and/or visual output including one or more flashes or patterns of flashes of light, and/or one or more colors or patterns of colors of light, generated by the sensory output device.

75. The medium of claim 71, wherein the sensory output intensifies or otherwise modulates as the sensor moves closer to the abnormality of interest.
